(19) United States
(12) Patent Application Publication
(10) Pub. No.: US 2010/0240040 A1
(43) Pub. Date: Sep. 23, 2010

(54) METHOD FOR SCREENING FOR SELECTIVE MODULATOR OF THE NF-KB PATHWAY ACTIVATION

(75) Inventors: Fabrice Agou, Paris (FR); Jeanne Chiaravalli, Paris (FR); Yves-Marie Coic, Meudon (FR); Francoise Baleux, Paris (FR); Alain Israel, Paris (FR); Michel Veron, Paris (FR)

Correspondence Address: OBLON, SPIVAK, MCCLELLAND MAIER & NEUSTADT, L.L.P., 1940 DUKE STREET, ALEXANDRIA, VA, 22314, US

(73) Assignees: INSTITUT PASTEUR, Paris (FR); Centre National De La Recherche Scient., Paris (FR)

(21) Appl. No.: 12/666,128

(22) PCT Filed: Jun. 20, 2008
(86) PCT No.: PCT/IB08/02633

§ 371 Date: Jun. 1, 2010

(30) Foreign Application Priority Data
Jun. 22, 2007 (EP) 07290782.7

(51) Int. Cl. C12Q 1/68; G01N 33/53; G01N 33/566

(52) U.S. Cl. 435/6; 435/7.1; 436/501

(57) ABSTRACT

The invention relates to methods for screening for selective modulator of NF-κB pathway activation. The invention concerns methods for primary screening molecules that potentially modulate (activate or inhibit) the NF-κB pathway activation by identifying and selecting molecules modulate the interaction of NEMO with other proteins. The invention also concerns methods for secondary screening for modulator (activator or inhibitor) of the NF-κB pathway activation.

The present invention relates to a method for identifying and selecting molecules that modulate (activate or inhibit) the NF-kB pathway activation by modulating the interaction of NEMO with other proteins.

Nuclear factor-κB (NF-κB) signaling is a signal transduction pathway involved in a variety of essential cellular processes including inflammatory responses, oncogenesis, viral infection, regulation of cell proliferation, apoptosis and antigenic stimulation of B and T lymphocytes (Ghosh, 1998, Annu. Rev. Immunol.; Karin, 1999, J. Biol. Chem.; Israel, 2000, Trends Cell Biol.; Santoro, 2003, EMBO J.). In mammalian cells, there are five NF-κB family members that dimerize: RelA, RelB, c-Rel, NF-κB2/p100/p52 and NF-κB1/p105/p50. NF-κB whose predominant form is a heterodimeric transcription factor composed of p50 and RelA subunits, remains, in resting cells, sequestered in the cytoplasm through their association with members of an inhibitory family of proteins known as IκB. Upon stimulation by several factors, including the cytokines TNF-α and the interleukin-1, endotoxin (LPS), microbial and viral infections, pro-inflammatory signals converge on the canonical IκB kinase complex (IKK), a protein complex that is composed of two kinases subunits, IKKα/IKK-1 and IKKβ/IKK-2 and a structural/regulatory subunit NEMO/IKK-γ. Once activated IKK complex phosphorylates IκB proteins, triggering their ubiquitination and subsequent degradation by proteasome. The released NF-κB transcription factors are then translocated into the nucleus to initiate or up-regulate gene expression. Although IKKα and IKKβ exhibit striking structural similarity (52%), genetic studies have shown that they are involved in two pathways for the activation of NF-κB (Pomerantz, 2002, Mol Cell). IKKβ is the pro-inflammatory kinase that is responsible for activation of classical NF-κB complexes whereas IKKα in association with NF-κB inducing kinase (NIK) plays an essential role in the non-canonical NF-κB signaling pathway (Senftleben, 2001, Science). IKKα plays also a role in keratinocyte differentiation but this process is independent of its kinase activity (Hu, 2001, Nature).

The NEMO protein (NF-κB essential modulator) plays a key role in the NF-κB pathway activation. The NEMO protein is associated with IKKα and IKKβ protein kinases in the IKK complex. The IKK kinases are activated by phosphorylation by an unknown mechanism, which is believed to be a result of NEMO oligomerization (Agou et al., 2004, J. Biol. Chem.). The presence of the NEMO protein underlies IKK activation since NEMO-deficient cells are unable to activate NF-κB in response to many stimuli.

The biochemical mechanisms triggering the activation of IKK in response to pro-inflammatory stimuli remain unclear. It has been demonstrated that phosphorylation on two serine residues in the activation T-loop induces activation of the IKKβ. However, the mechanism that leads to this phosphorylation event is still unknown. One possible mechanism consists of the conformation change of the kinase induced by NEMO oligomerization (Agou et al., 2004, J. Biol. Chem.). This change of the oligomeric state may induce the T-loop activation by a mechanism of trans-autophosphorylation (Zandi, 1997, Cell; Tang, 2003, J. Biol. Chem.).

Consistent with the role of NEMO oligomerization in IKK activation, mutations in the minimal oligomerization domain failed to rescue NF-κB by genetic complementation in NEMO-deficient cells activation in responses to many stimuli. Moreover, enforced oligomerization of NEMO leads to full activation of the IKK complex. (Inohara, 2000, J. Biol. Chem.; Poyet, 2000, J. Biol. Chem.; Poyet, 2001, J. Biol. Chem.). Recently, the phosphorylation and the ubiquitination of NEMO in response to TNF-α have been reported, (Carter, 2001, J. Biol. Chem.; Trompouky, 2003, Nature; Kovalenko, 2003, Nature). However, these NEMO modifications have not been demonstrated yet as a crucial step in the activation of the IKK complex in response to several pro-inflammatory stimuli.

Inhibition of NF-κB activation constitutes a privileged target for development of new anti-inflammatory and anti-cancer drugs (May, 2000, Science; Poulaki, 2002, Am J Pathol). Among many protein actors in the NF-κB signaling pathway, the IKK complex represents one of the most promising molecular targets for discoveries of new specific NF-κB inhibitors. To minimize the potential toxic effects in vivo, therapeutical success will greatly depend on the abilities of the NF-κB inhibitors to block activating signals without modifying the basal level of NF-κB activity. May et al. described a cell-permeable peptidic inhibitor that blocks specifically pro-inflammatory NF-κB activation by disrupting the constitutive NEMO interaction with IKK kinases (May, 2000, Science; May, 2002, J. Biol. Chem.). Modulating protein-protein interactions by the rational design of peptides that alter a protein's function provides an important tool for both basic research and development of new classes of therapeutic drugs (Souroujon, 1998, Nat. Biotechnol.), especially with signaling proteins that exhibit flexible and dynamic binding properties (Pawson, 2003, Science).

Numerous studies of peptide modulators have been described in the literature where peptides mediate protein's function by interfering with localization (translocation) (Lin, 1995, J. Biol. Chem.), recruitment to receptor (Chang, 2000, J. Biol. Chem.), intramolecular interactions (Souroujon, 1998, Nat. Biotechnol.) and oligomerization (Judice, 1997, P.N.A.S.). In the latter, inhibition of HIV-1 gp41 fusion protein with various peptides provides a clear proof-of concept (for a review see Chan, 1998, Cell and Eckert, 2001, Ann. Rev. Biochem.).

Therefore, the NEMO protein is a promising target for the development of new drugs inhibiting the NF-κB pathway, since it integrates and coordinates most of NF-κB stimuli and it is a non redundant component of the IκB Kinase complex (IKK).

The amino acid sequence of NEMO protein suggests that this protein consists of several domains (Agou et al., J. Biol. Chem., 2004b). Briefly, the N-terminal part of the polypeptide contains a large coiled-coil motif (CC1) and all the residues involved in the interaction of the protein with the IKK kinases (IKK-binding domain). The C-terminal half (residues 250-412) is composed of two successive coiled-coil motifs, CC2 (residues 253-285) and LZ (residues 301-337), and a zinc finger motif (ZF) at the extreme C-terminus of the protein and functions as the regulatory part of the protein, which has often been reported as a binding template to link many upstream signaling molecules or viral proteins (Ghosh, 1998, Annu. Rev. Immunol.; Santoro, 2003, EMBO J.).

Interestingly, mutations responsible for incontinentia pigmenti (IP) and ectodermal dysplasia with immunodeficiency (EDA-ID) were mainly found in this part of the molecule (Döffinger, 2001, Nature Gen.; Zonana, 2000, Am. J. Hum. Genet). The minimal oligomerization domain (MOD) of NEMO consists of the CC2 and LZ coiled-coil motifs (Agou et al., J. Biol. Chem., 2004a). The MOD domain also includes a NLM motif (Nemo Like Motif) (293-322) (Agou et al., 2004b). The residues numeration indicated above is relative to the murine NEMO protein of sequence SEQ ID NO: 1. With respect to the human NEMO protein, CC2, LZ and NLM domains respectively correspond to residues 260-292, residues 301-344 and residues 300-329 of the sequence SEQ ID NO: 2.

The Inventors have previously synthesized peptides, derived from the MOD domain of NEMO, which are able to inhibit the NF-κB pathway by inhibiting the NEMO oligomerization (Patent Application WO 2005/027959). Indeed, spLZ peptides of sequences SEQ ID NO: 6 and SEQ ID NO: 7, which respectively correspond to the LZ domain of the murine and the human NEMO proteins, and spCC2 peptides of sequences SEQ ID NO: 8 and SEQ ID NO: 9, which respectively correspond to the CC2 domain of the murine and the human NEMO proteins, inhibit NF-κB activation with an IC₅₀ in the μM range (WO2005/027959, Agou et al., 2004b).

Other peptides, NLM and DR-NLM, deriving from the N-terminal sequence of the LZ motif of NEMO also inhibit NF-κB activation. Their IC₅₀ values were reported in the International PCT Application WO 2005/027959. The DR-NLM peptide is half the length of the LZ peptide (International PCT Application WO 2005/027959). Peptides NLM (SEQ ID NO: 10) and DR NLM (SEQ ID NO: 11) correspond to residues 294-314 of SEQ ID NO: 1 and differ in that in DR NLM the Asp residue in position 304 of SEQ ID NO: 1 is substituted with a Arg residue. NLM and DR NLM derived from human NEMO protein are respectively defined by SEQ ID NO: 18 and SEQ ID NO: 12.

The methods of W0 2005/027959 and Agou et al., 2004b both use in vivo cell based assay systems to determine whether or not a particular substance affects the Nuclear factor-κB pathway. In particular they use a NF-κB inhibition assay using an endogenous NF-κB-lacZ reporter gene to determine the effects of different peptides, such as the CC2 or LZ domain, upon the NF-κB pathway. Such in vivo cell based assays are known to have a number of drawbacks particularly due to the complexity of maintaining suitable cell lines and ensuring all relevant factors affecting the assay remain within the required parameters. The large scale screening of compounds using such in vivo assays is therefore both time consuming, complex and expensive in comparison to in vitro screening techniques. Unexpectedly, the Inventors have now found that the MOD domain acts as oligomerization domain as well as polyubiquitin chain binding domain. These dual properties of said NEMO domains allow to search for new compounds modulating specifically the NF-κB signaling pathway by modulating (enhancing or disrupting) NEMO oligomerization or by modulating (enhancing or disrupting) the interaction between NEMO and the polyubiquitin chain. Indeed, whilst the LZ specifically inhibits the NF-κB activation by disrupting NEMO oligomerization, the DR NLM acts as NF-κB inhibitor by blocking the interaction between the polyubiquitin chains and the NEMO protein. These results also provide new insights into the NLM domain of NEMO which corresponds to a new and crucial ubiquitin binding domain required for the NEMO function. More importantly, these results allow to define the “hot spot” region of NEMO which could be used as a target for virtual screening.

NEMO-polyubiqutin interactions have been described before; Godha et al., (2007) describes an in vivo study into the mechanism of how Human T-cell Leukaemia Virus type 1 (HTLV-1) activates the NF-κB pathway. The authors show that a MAP kinase TAK1 induces K63 polyubiquitination of NEMO which they believe to be at least one of the mechanisms which HTLV-1 uses to affect the NF-κB pathway.

Also Zhou et al., (2004) describes a study into the effects of Bcl10 over-expression upon the NF-κB pathway. In particular they show that Bcl10 over-expression targets NEMO for K63 polyubiquitination and use an in vitro ubiquitination assay to demonstrate this.

Both Godha et al., (2007) and Zhou et al., (2004) use full length NEMO as a polyubiquitin target.

The Inventors however have found in their work that a peptide comprising just the essential CC2 and LZ domains of NEMO can be used in methods to identify modulators of NEMO activity. By using only this essential portion of NEMO, this allows more sensitive assays to be conducted to find modulators interacting with these most important portions of NEMO and ubiquitin, rather than more general interactions with other portions of NEMO which may not have any specific function.

Considering the role of NEMO in inflammatory response, oncogenesis and viral infection and the complexity of the NF-κB signaling pathway, there is a need of primary screening of substances able to act by disrupting any NEMO interaction with other proteins (i.e. oligomerization or interaction between NEMO and the polyubiquitin chain).

Therefore, the purpose of the present invention is to provide a screening assay for selecting molecules that specifically modulate (activate or inhibit) the interaction of NEMO with other proteins. More specifically, said screening assay uses the MOD domain of the NEMO, for detecting new efficient and specific modulators of the NF-κB pathway activation.

Accordingly, a first object of the present invention is an in vitro method for primary screening for a potential modulator of the NF-κB pathway activation, characterized in that it comprises the following steps:

(a) bringing into contact (i) a peptide P1 consisting of the CC2 and LZ regions of NEMO, said peptide P1 being labelled or not, and (ii) a peptide P2, labelled or not, selected from the group consisting of a peptide comprising at least 10 amino acid residues of the LZ region of NEMO, preferably at least 36 amino acid residues of the LZ region of NEMO, a peptide comprising at least 10 amino acid residues of the NLM region of NEMO, preferably at least 21 amino acid residues of the NLM region of NEMO, a peptide comprising DR-NLM, a peptide comprising the regions CC2 and LZ of NEMO, a peptide comprising at least 10 amino acids residues of the CC2 region of NEMO, and a peptide comprising a K63-linked polyubiquitinylated chain; in the absence of the substance S to be tested;

(b) bringing into contact said peptide P1, labelled or not, and said peptide P2, labelled or not, in the presence of the substance S to be tested;

(c) detecting the complexes between P1 and P2 obtained in step (a) by measuring an appropriate signal;

(d) detecting the complexes between P1 and P2 obtained in step (b) by measuring an appropriate signal;

(e) comparing the signal measured in (c) and the signal measured in (d) and selecting the substance S for which the ratio of the signal measured in (c)/signal measured in (d) is different from 1.

According to the present invention, the terms region(s) and domain(s) are interchangeable. The CC2 and LZ regions (or domains) of NEMO are as described above.

Preferably, P1 is selected from the group consisting of the peptide sp CC2-LZ of SEQ ID NO: 17, the peptide spCC2-LZ of SEQ ID NO: 19, the murine NEMO protein of SEQ ID NO: 1, the human NEMO protein of SEQ ID NO: 2, the biotin labelled CC2-LZ peptide of SEQ ID NO: 28, the biotin labelled humanised CC2-LZ peptide of SEQ ID NO: 29, the six his-tagged CC2-LZ peptide of SEQ ID NO: 30 and the six his-tagged humanised CC2-LZ peptide of SEQ ID NO: 31.

Preferably, P2 is selected from the group consisting of the peptides of sequences SEQ ID NO: 1, SEQ ID NO: 2, SEQ ID NO: 6 to 12, SEQ ID NO: 17 to 19, SEQ ID NO: 28 to 31.

The peptides used in the method according to the invention can be synthesized as described in the Patent Application WO 2005/027959 and in Agou et al., 2004b.

The prefix “sp” (synthetic polypeptide) means that said peptide is synthetic and may be used for instance to distinguish a peptide (e.g. spCC2) from the corresponding region of the NEMO protein (e.g. CC2 region). However, peptide and sp peptide refer to the same product.

Furthermore, without precision, the different peptides relate indifferently to the murine NEMO protein and to the human NEMO protein.

Preferably, said K63-linked polyubiquitinylated molecule is selected from the group consisting of a K63-linked polyubiquitine chain, for example K63Ub₂₋₇ (Boston Biochem, Inc), a protein coupled to a K63-linked polyubiquitine chain, such as the polyubiquitinylated protein RIP1 (Receptor Interacting Protein kinase 1) (Ea et al., 2006), and a cell extract comprising K63-linked polyubiquitinylated proteins, such as a cell extract from JM4.5.2 cells which have been previously stimulated with TNF-α.

In a preferred embodiment of said method, said peptide P1 is immobilized on a solid support. Preferably, said solid support consists of magnetic beads. Advantageously, the peptide P1 is coupled to a biotinyl group and is immobilized on streptavidin magnetic beads.

Preferably, the signal in steps (c) and (d) is measured by Western blot, by fluorescent anisotropy, by Fluorescent Resonance Energy Transfer (FRET) or by Homogeneous Time Resolved Fluorescence (HTRF).

In a preferred embodiment of said method, said peptide P1 is unlabelled, and said peptide P2 is labelled with a fluorescent marker M1.

Preferably, said marker M1 is selected from the group consisting of: green fluorescent protein (GFP), cyan fluorescent protein, yellow fluorescent protein and a fluorophore comprising a maleimide group. Other examples of fluorescent markers are described in Shaner et al. (Nat. Biotech., 2004, 22, 1567). Other fluorophores that can be used include CFP/ds Red and/or GFP/ds Red (Erickson et al., Biophys J., 2003, 85, 599-611) and Cyan variants of the orange type (Karawawa et al., Biochem J., 2004, 381(Pt1), 307-12). Example of a fluorescent marker comprising a maleimide group is the Bodipy®FL N-(2-aminoethyl) maleimide fluorophore (Molecular Probe).

Preferably, said labelled peptide P2 is selected from the group consisting of the peptides of sequences SEQ ID NO: 4, 5 and 13 to 16.

In further another preferred embodiment of said method, said peptide P1 is labelled with a marker M2 and said peptide P2 is labelled with a marker M3, M2 and M3 being a couple of a fluorophore donor and a fluorophore acceptor.

Examples of couples of fluorophore donor-fluorophore acceptor are the couple Europium cryptate-XL665 (Cis-Bio) and the couple Cy5/5 (GE Healthcare)-BHQ3 (Biosearch Technologies).

Preferably, a hybrid construct is used such that the peptide P1 and/or P2 is linked, either directly or through a linker molecule (e.g., a short peptide), to the fluorescent protein. The linkage can occur at the N-terminus or C-terminus of the peptide, preferably provided that the added linkage does not interfere with the binding between P1 and P2.

The interaction between P1 and substances that modulate (activate or inhibit) its binding to P2 can be detected for instance using the FRET method, by direct measurement of the fluorescence emission of the fluorophore acceptor resulting from the proximity of the two different markers (fluorophore donor and fluorophore acceptor). A decrease in the fluorescence emission of the fluorophore acceptor in the presence of the substance S, compared to the fluorescence emission of said fluorophore acceptor in the absence of said substance S, indicates that said substance S interferes with the binding of P1 to P2. Hence, a decrease in said fluorescent emission indicates that said substance S interferes with the binding of P1 to P2, while an increase in said fluorescent emission indicates that said substance S enhances said binding of P1 to P2.

Advantageously, M2 consists of a first tag and an antibody directed against said first tag and labelled with a fluorophore donor, and M3 consists of a second tag and an antibody directed against said second tag and labelled with a fluorophore acceptor, said two tags being different.

Preferably, said tag of the marker M2 is a poly His sequence, and said tag of the marker M3 is biotin.

In a further preferred embodiment of said method, said potential modulator is a potential activator and step (e) comprises comparing the signal measured in (c) and the signal measured in (d) and selecting the substance S for which the ratio of the signal measured in (c)/signal measured in (d) is less than 1.

In another preferred embodiment of said method, said potential modulator is a potential inhibitor and step (e) comprises comparing the signal measured in (c) and the signal measured in (d) and selecting the substance S for which the ratio of the signal measured in (c)/signal measured in (d) is greater than 1.

In the case where the peptide P2 represents a peptide comprising a K63-linked polyubiquitine chain, the peptide P1 is advantageously selected from the group consisting of a peptide comprising at least 10 amino acid residues of the NLM region of NEMO, preferably at least 21 amino acid residues, and a peptide comprising DR NLM. Preferably, said peptide P1 is selected from the group consisting of the peptides of sequences SEQ ID NO: 1, 2, 10, 17 to 19 and 28 to 31.

Thus, in such a case, the method for primary screening for a potential modulator of the NF-κB pathway activation, is characterized in that it comprises the following steps:

(a) bringing into contact (i) a peptide P1 selected from the group consisting of a peptide comprising at least 10 amino acid residues of the NLM region of NEMO, preferably at least 21 amino acid residues, and a peptide comprising DR NLM, said peptide P1 being labelled or not, and (ii) a peptide P2 comprising a K63-linked polyubiquitine chain, said peptide P2 being labelled or not, in the absence of the substance S to be tested;

(b) bringing into contact said peptide P1, labelled or not, and said peptide P2, labelled or not, in the presence of the substance S to be tested;

(c) detecting the complexes between P1 and P2 obtained in step (a) by measuring an appropriate signal;

(d) detecting the complexes between P1 and P2 obtained in step (b) by measuring an appropriate signal;

(e) comparing the signal measured in (c) and the signal measured in (d) and selecting the substance S for which the ratio of the signal measured in (c)/signal measured in (d) is different from 1.

Preferably, said peptide P1 is selected from the group consisting of the peptides of sequences SEQ ID NO: 1, 2, 10, 17 to 19 and 28 to 31.

Preferably, said peptide P2, comprising a K63-linked polyubiquitinylated chain, is immobilised on a solid support, as described above.

Preferably, said peptide P2 is labelled with a fluorescent marker M1 as described here above and the signal in steps (c) and (d) is measured as described above.

In a preferred embodiment of said method, said potential modulator is a potential activator and step (e) comprises comparing the signal measured in (c) and the signal measured in (d) and selecting the substance S for which the ratio of the signal measured in (c)/signal measured in (d) is less than 1.

In another preferred embodiment of said method, said potential modulator is a potential inhibitor and step (e) comprises comparing the signal measured in (c) and the signal measured in (d) and selecting the substance S for which the ratio of the signal measured in (c)/signal measured in (d) is greater than 1.

Alternatively, the method for primary screening for a potential modulator of the NF-κB pathway activation, comprises the following steps:

(a) bringing into contact a peptide P1 comprising the CC2 and LZ regions of NEMO with the substance S to be tested, said peptide P1 being labelled with the markers M2 a fluorophore donor and M3 a fluorophore acceptor, i.e, being coupled to both a fluorophore donor and to a fluorophore acceptor;

(b) measuring the fluorescence emission in the absence of the substance S to be tested;

(c) measuring the fluorescence emission in the presence of the substance S to be tested;

(d) comparing the signal measured in (b) and the signal measured in (c) and selecting the substance S for which the ratio signal measured in (b)/signal measured in (c) is different from 1.

Preferably, said signal in steps (c) and (d) is measured by FRET or by HTRF as described above.

Advantageously, said fluorophore donor is coupled to the N-terminus of the CC2 region, and said fluorophore acceptor is coupled to the C-terminus of the LZ region.

In a preferred embodiment of said alternative method, said potential modulator is a potential activator and step (d) comprises comparing the signal measured in (b) and the signal measured in (c) and selecting the substance S for which the ratio of the signal measured in (b)/signal measured in (c) is less than 1.

In another preferred embodiment of said alternative method, said potential modulator is a potential inhibitor and step (d) comprises comparing the signal measured in (b) and the signal measured in (c) and selecting the substance S for which the ratio of the signal measured in (b)/signal measured in (c) is greater than 1.

Alternatively, the method for primary screening for a potential modulator of NF-κB pathway activation is characterized in that it comprises the following steps:

a) providing at least one cell deficient in endogenous NEMO, which is transformed with one or more polynucleotides that express at least a peptide P1 and a peptide P2 as defined in the first object of the present invention;

b) applying the substance S to the at least one cell;

c) measuring the formation of complexes between P1 and P2 in the absence of the substance S by measuring an appropriate signal;

d) measuring the formation of complexes between P1 and P2 in the presence of the substance S by measuring an appropriate signal;

e) the signal measured in (c) and the signal measured in (d) and selecting the substance S for which the ratio of the signal measured in (c)/signal measured in (d) is different from 1.

In a preferred embodiment of this alternative method, P1 is a fusion protein GFP-NEMO and P2 is a fusion protein Flag-NEMO, Flag being a hydrophilic 8 amino acid residues peptide (SEQ ID NO: 23). Preferably, P1 is defined by the sequence SEQ ID NO: 27 and P2 is defined by the sequence SEQ ID NO: 25.

In another preferred embodiment of this alternative method, the steps (c) and d) comprise:

c1) (or d1)) the cell lysis,

c2) (or d2)) the immunoprecipitation of Flag-NEMO, bound or not to GFP-NEMO, using antibody directed against the Flag moiety of Flag-NEMO, and

c3) (or d3)) the detection of the fluorescence emission of GFP moiety of GFP-NEMO bound to the immunoprecipitated Flag-NEMO.

In a preferred embodiment of said alternative method, said potential modulator is a potential activator and step (e) comprises comparing the signal measured in (c) and the signal measured in (d) and selecting the substance S for which the ratio of the signal measured in (c)/signal measured in (d) is less than 1.

In another preferred embodiment of said alternative method, said potential modulator is a potential inhibitor and in that step (e) comprises comparing the signal measured in (c) and the signal measured in (d) and selecting the substance S for which the ratio of the signal measured in (c)/signal measured in (d) is greater than 1.

Another object of the present invention is a method for secondary screening for an inhibitor of NF-κB pathway activation, comprising:

a) selecting a substance S potentially able to inhibit NF-κB pathway activation by any method for primary screening for a potential inhibitor according to the invention as described above,

b) applying an activator of NF-κB pathway in the absence or in the presence of the substance S selected in a), to a cell comprising a reporter gene under the control of a promoter inducible by NF-κB;

c) detecting the expression of the reporter gene by measuring an appropriate signal in the absence of the substance S;

d) detecting the expression of the reporter gene by measuring an appropriate signal in the presence of the substance S; and

e) comparing the signal measured in (c) and the signal measured in (d) and selecting the substance for which the ratio of the signal measured in (c)/signal measured in (d) is greater than 1.

Preferably, said activator of NF-κB pathway used in step c) is selected from the group consisting of: TNF-α, IL-1β, PMA, ionomycin, and LPS from Salmonella abortus.

The reporter gene is preferably selected from the group consisting of the lacZ gene, encoding the βgalactosidase enzyme, and the luc gene, encoding the luciferase. The reporter gene is preferably under the control of a promoter comprising the SRE sequence (Serum Response Element). Such constructs are described in the Patent Application WO2005/027959 and in Agou et al., 2004b.

In a preferred embodiment of this method for secondary screening, said cell is a 70Z/3-C3 cell, deposited in the Collection Nationale de Cultures de Microorganismes (C.N.C.M.) on Apr. 1, 2003, under the number I-3004.

In another preferred embodiment, said cell is a T lymphocyte or a MEF cell (Murine embryonic Fibroblast) comprising a reporter system for the NF-κB pathway.

It is preferred that said inhibitor of the NF-κB pathway activation, selected by the methods according to the invention, is a specific inhibitor of the NF-κB pathway which does not inhibit the ERK and p38 pathway activation, the NF-AT pathway activation, and the AP1 pathway activation.

Therefore the secondary method for screening advantageously comprises the additional steps of determining whether the selected inhibitor acts on the ERK and p38 pathway, on the NF-AT pathway and on the AP1 pathway. The inhibitors which are selected by said method are those which do not inhibit ERK, p38, NF-AT and AP1 pathways activation.

Assaying the inhibition of the ERK and p38 pathway, the NF-AT pathway and the AP1 pathway may be carried out for instance using a reporter gene placed under the control of a promoter inducible by the corresponding pathway. Examples of reporter genes are described above. Examples of constructs inducible by the ERK and p38 pathway, the NFAT pathway, and the AP1 pathway are respectively SRE-luc (Courtois et al., 1997, Mol. Cell. Biol, 17:1441-1449), NF-AT-luc and AP1-luc (Northrop et al., 1993, J. Biol. Cell., 268:2917-2923). Expression of the reporter gene is assayed for example by using transformed cells, such as comprising the corresponding construct, stimulated by PMA and ionomycin.

Another object of the present invention is a method for secondary screening for an activator of NF-κB pathway activation, comprising:

a) selecting a substance S potentially able to activate NF-κB pathway activation by a method for primary screening for a potential activator according to the invention as described above,

b) providing a cell comprising a reporter gene under the control of a promoter inducible by NF-κB;

c) detecting the expression of the reporter gene by measuring an appropriate signal in the absence of the substance S;

d) detecting the expression of the reporter gene by measuring an appropriate signal in the presence of the substance S; and

e) comparing the signal measured in (c) and the signal measured in (d) and selecting the substance for which the ratio of the signal measured in (c)/signal measured in (d) is less than 1.

The reporter gene is preferably selected from the group consisting of the lacZ gene, encoding the β-galactosidase enzyme, and the luc gene, encoding the luciferase. The reporter gene is preferably under the control of a promoter comprising the SRE sequence (Serum Response Element) as described above.

In a preferred embodiment of this method for secondary screening, said cell is a 70Z/3-C3 cell, deposited in the Collection Nationale de Cultures de Microorganismes (C.N.C.M.) on Apr. 1, 2003, under the number I-3004.

A more complete appreciation of the invention and many of the attendant advantages thereof will be readily obtained as the same becomes better understood by reference to the following Figures in conjunction with the detailed description below.

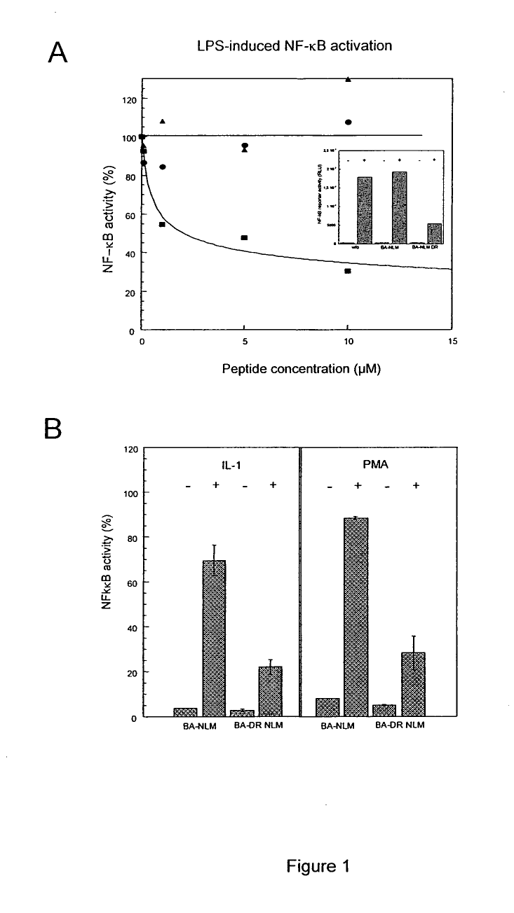

FIG. 1: Inhibition of NF-κB activation by the peptide DR NLM. A, Stably transfected 70Z/3-C3 cells were incubated with bodipy tagged Antennapedia (BA) (black triangles), BA-NLM (filled circles) and BA-DR NLM (filled squares) (0-10 μM) for 2 h before activation with 15 μg/ml LPS for 4 h. NF-κB activity was measured using the β-galactosidase (β-Gal) assay. Inset, NF-κB activity after incubation with or without (w/o) 10 μM of BA-NLM or BA-DR NLM peptides with (+) or without (−) LPS activation. B, 70Z/C3 cells were incubated for 2 h with 10 μM of BA-NLM or BA-DR NLM peptides. Cells were treated for 5 h with (+) or without (−) PMA or IL-1. NF-κB activity was measured using the β-galactosidase assay.

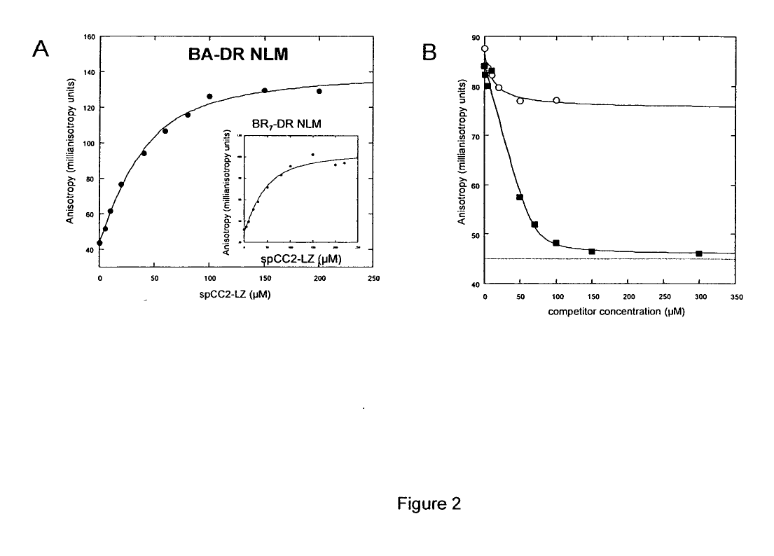

FIG. 2: In vitro binding of the peptide BA-DR NLM to the peptide spCC2-LZ. A, Analysis of the formation of BA-DR NLM:spCC2-LZ complexes using fluorescent anisotropy. Inset: analysis of the formation of complexes between BR₇-DR NLM and spCC2-LZ using fluorescent anisotropy. B, Determination of the binding site of BA-DR NLM to spCC2-LZ by competition assay using fluorescence anisotropy. Fluorescent BA-DR NLM (10 μM) was incubated with spCC2-LZ (30 μM) to form the BA-DR NLM:spCC2-LZ complex. Increasing concentrations of A-NLM (open circles) and of the C-terminal part of LZ fused to the antennapedia sequence (A-Cterm-LZ) (filled squares) were then added to displace the BA-DR NLM bound to spCC2-LZ. The dashed line represents the anisotropy level of free BA-DR NLM.

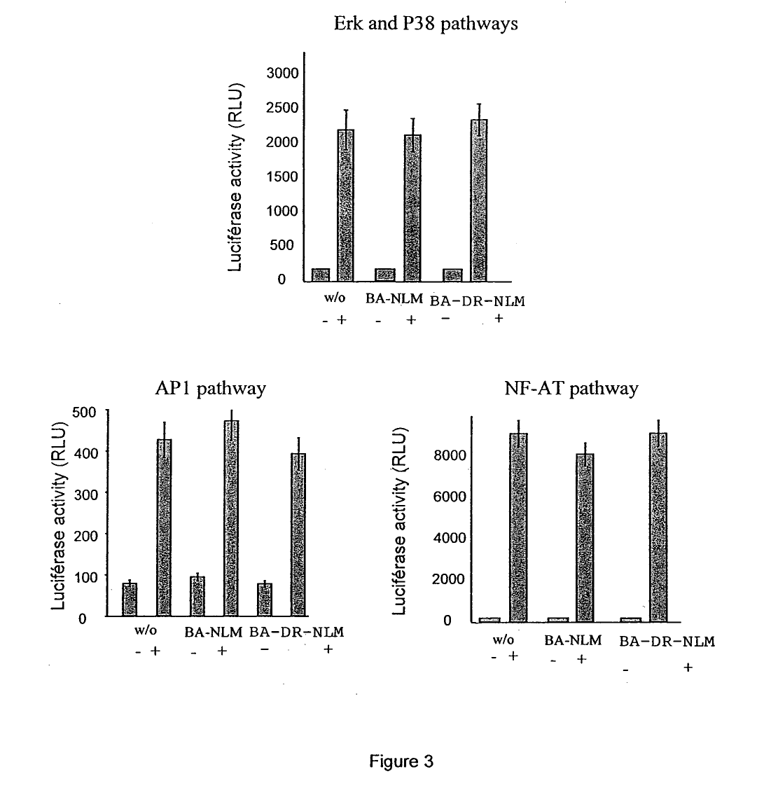

FIG. 3: the peptide DR NLM does not affect other signalling pathways. Inhibition effect of BA-NLM and BA-DR NLM on the AP1, Erk and P38 MAPK pathways and the NFAT pathway. Jurkat cells transiently transfected with different reporter plasmids (SRE-luc, AP1-luc, NFAT-luc) were incubated with or without (w/o) 5 μM of BA-NLM or BA-DR NLM for 2 h and then mock-stimulated (−) or stimulated (+) with PMA (100 ng/ml) and ionomycin (1 μg/ml) for 5 h.

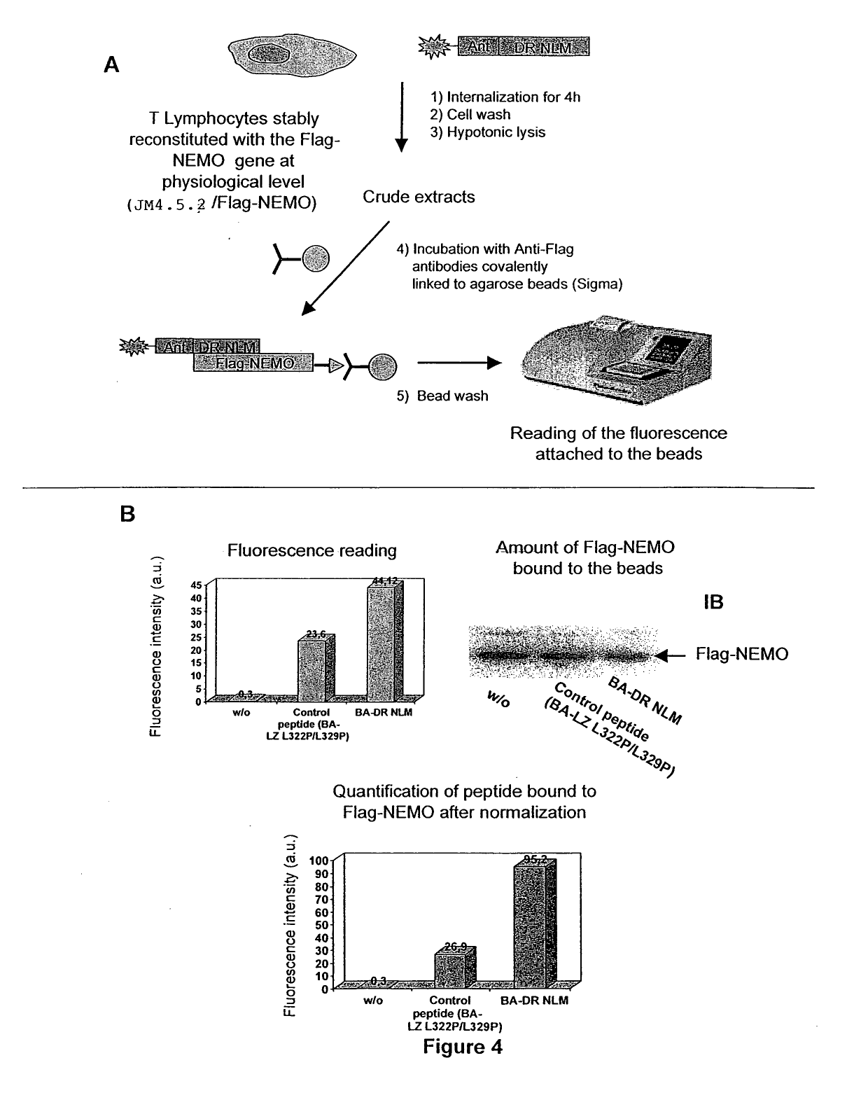

FIG. 4: the peptide DR NLM interacts with the endogenous NEMO. A, overview of the method for identifying the protein target of DR NLM peptide. B, Fluorescence measurements of Flag-NEMO coated beads. T lymphocytes stably reconstituted with Flag-NEMO (JM4.5.2/Flag-NEMO) were treated in the absence (w/o) or in the presence of the indicated fluorescent peptides for 2 h to allow peptide internalization. The control peptide (BA-LZ L322P/L329P) comprises the sequence of the wild type NLM. Following several cell washes to remove any peptide excess, the cells were lysed and the endogenous Flag-NEMO was pulled down using anti-Flag antibodies. The amount of peptide bound to NEMO was then examined by fluorescence (top left). To normalize all samples, the amount of NEMO coated to the beads was determined by Western blotting using anti-NEMO antibodies (top right) was used to normalize all samples (bottom).

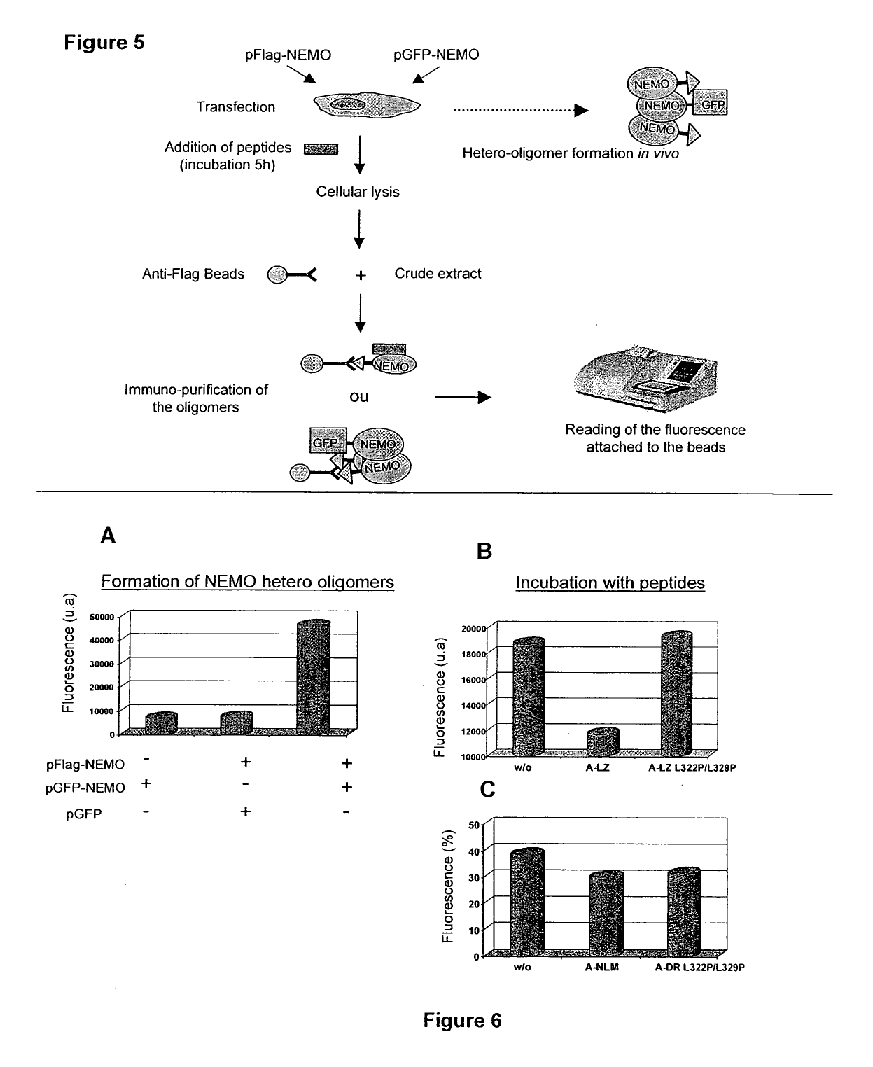

FIG. 5: Schematic view of the cell based assay (cell-based assay) to probe the effects of different peptides on NEMO oligomerization.

FIG. 6: DR NLM peptide does not inhibit NEMO oligomerization in cells. A, formation of hetero-oligomers with GFP-NEMO and FLAG-NEMO. B, C, Co-tranfected 293T cells with GFP-NEMO and Flag-NEMO were treated for 2 h with 20 μM of the peptides A-LZ, A-LZ L322P/L329P (B), A-NLM or A-DR NLM (C). The NEMO hetero oligomers were then immunopurified and the amount of the fluorescent NEMO subunit bound to hetero oligomers was examined by fluorescence as described in Example 1.I.

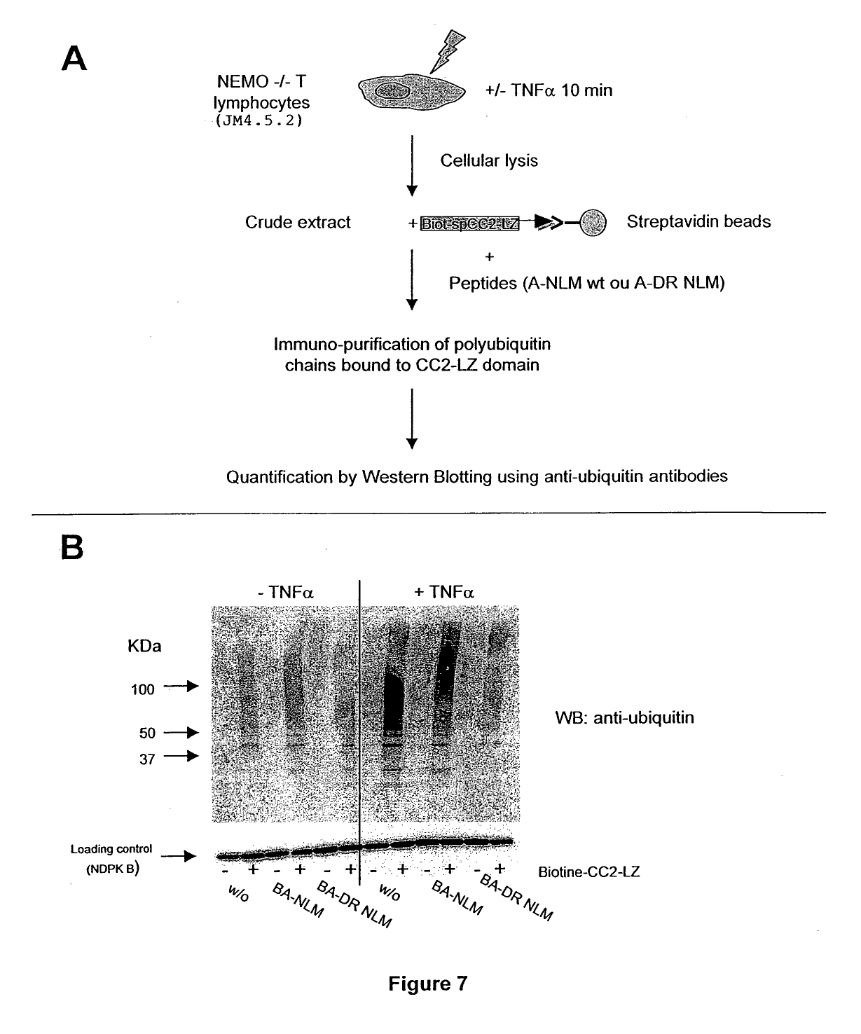

FIG. 7: DR NLM impedes the interaction of cellular polyubiquitinylated proteins with the peptide CC2-LZ comprising the CC2 and LZ domains of NEMO. A, overview of the method for probing the interaction of polyubiquitinylated proteins to the peptide spCC2-LZ. B, Western blotting of polyubiquitinylated proteins binding to the peptide spCC2-LZ with or without stimulation by TNF-α, and with (+) or without (−) the peptide BA-NLM or BA-DR NLM.

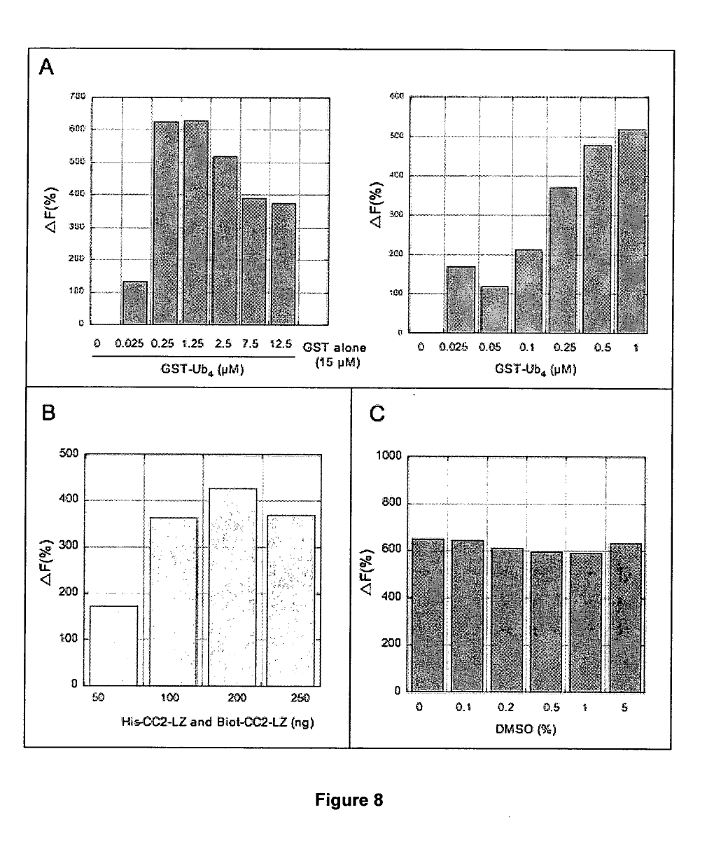

FIG. 8: Optimizations of the HTRF assay. A, Determination of the optimal amount of GST-Ub4. Biot-CC2-LZ and His-CC2-LZ (50 nM each) were mixed each other in the binding buffer (20 mM KPO₄, 100 mM KF and 0.1% BSA, pH 7) and then incubated with Anti-His-XL665 (7 nM) and Streptavidin-EuK (2 nM). Following 5 min incubation, GST-Ub₄ was added at different concentrations. FRET signal was monitored after 1 h incubation. B, Determination of the optimal amount of His-CC2-LZ and Biot-CC2-LZ. Total concentration of Biot-CC2-LZ and His-CC2-LZ with a stoichiometric ratio of 1 to 1 were incubated together in the binding buffer, and then mixed with Anti-His-XL665 (7 nM), Streptavidin-EuK (2 nM) and GST-Ub₄ (250 nM). FRET signal was recorded after 1 h incubation. C, The effect of DMSO on fluorescence signal. Experiments were similar to A using a GST-UB₄ concentration of 250 nM, and with the exception that different concentrations of DMSO were used in the binding buffer.

There will now be described by way of example a specific mode contemplated by the Inventors. In the following description numerous specific details are set forth in order to provide a thorough understanding. It will be apparent however, to one skilled in the art, that the present invention may be practiced without limitation to these specific details. In other instances, well known methods and structures have not been described so as not to unnecessarily obscure the description.

### EXAMPLES

### Example 1

### Materials and Methods

### A.—Cell Culture, Biological Reagents and Antibodies

Absence of endotoxin contamination was checked in the RPMI 1640 medium (Invitrogen) and in the fetal calf serum (Biowest) used in cell culture. Jurkat (clone 20) cells were maintained in RPMI 1640 supplemented with 10% fetal calf serum. Stable cell lines (70Z/3-C3, C.N.C.M. I-3004) were obtained after electroporation of 70Z/3 cells in the presence of a cx12lacZ-κB plasmid containing three tandem copies of an NF-κB binding site in the interleukin-2 promoter upstream from the lacZ reporter gene as described in Agou et al., 2004b. They were maintained in RPMI 1640 supplemented with 10% fetal calf serum and 50 μM β-mercaptoethanol (Sigma). Stable cell lines (JM4.5.2/Flag-NEMO) were obtained after electroporation of NEMO deficient T lymphocytes (JM4.5.2, Harhaj et al. 2000) in the presence of a pcDNA3 plasmid containing the sequence of murine Flag-NEMO (Flag is an hydrophilic 8 amino acid peptide of sequence SEQ ID NO: 23 provided by Sigma). These human Jurkat leukemic T-cells were maintained in RPMI 1640 supplemented with 10% fetal calf serum.

The human embryonal kidney cell line 293T (American Type Culture Collection; Manassas, Va.) was grown in DMEM medium (Invitrogen) supplemented with 100 units/ml penicillin and streptomycin (Invitrogen) and 10% fetal calf serum. LPS from Salmonella abortus, phorbol 12-myristate 13-acetate (PMA) and ionomycin were purchased from Sigma and the recombinant mouse IL-1β was purchased from BD Pharmingen. The polyclonal rabbit anti-NEMO serum was a gift from R. Weil and the specific antibodies were purified by immunoaffinity. The anti-GFP polyclonal antibody was from Oncogene. The anti-Flag M2 monoclonal antibody as well as the anti-ubiquitin antibody were from Sigma.

### B.—Peptide Synthesis and Purification

Peptides are synthesized as previously described (Agou et al., 2004b). Sequences of peptides were already reported in the International Patent WO2005027959 and some of them are represented in the following Table I.

TABLE I
Sequence of NEMO derived peptides

| | | SEQ | Theoretical | Experimental | Constructions | SEQ |
|---|---|---|---|---|---|---|
| Name | Sequence⁽¹⁾ | ID NO: | mass (Da) | mass (Da) | with NEMO Human sequences | ID NO: |
| BODIPY-Ant | B-CRQIKIWFQNRRMKWKK | 3 | 2805.19 | 2805.06 ± 0.52 | | |
| (BA) | | | | | | |
| BA-LZ | B-CRQIKIWFQNRRMKWKKLK | 4 | 8064.2 | 8063.9 ± 0.48 | B-CRQIKIWFQNRRMKWKKLKAQADIYKA | 5 |
| | AQADIYKADFQAERHAREKLV | | | | DFQAEQAREKLAEKKELLQEQLEQLQREY | |
| | EKKEYLQEQLEQLQREFNKL | | | | SKL | |
| LZ | LKAQADIYKADFQAERHAREK | 6 | 5318.08 | 5318.21 ± 0.5 | LKAQADIYKADFQAERQAREKLAEKKELL | 7 |
| | LVEKKEYLQEQLEQLQREFNK | | | | QEQLEQLQREYSKL | |
| | L | | | | | |
| CC2 | SKGMQLEDLRQQLQQAEEALV | 8 | 4155.75 | 4155.86 +/− 0.53 | SKGMQLEDLKQQLQQAEEALVAKQEVIDK | 9 |
| | AKQELIDKLKEEAEQHKIV | | | | LKEEAEQHKIV | |
| NLM | LKAQADIYKADFQAERHAREK | 10 | | | LKAQADIYKADFQAERQAREK | 18 |
| DR NLM | LKAQADIYKARFQAERHAREK | 11 | 2570.94 | 2570.78 +/− 0.21 | LKAQADIYKARFQAERQAREK | 12 |
| BA-DR NLM | B-CRQIKIWFQNRRMKWKKLK | 13 | 5317.08 | 5316.38 +/− 0.26 | B-CRQIKIWFQNRRMKWKKLKAQADIYKA | 14 |
| | AQADIYKARFQAERHAREK | | | | RFQAERQAREK | |
| BR₇-DR NLM | B-CRRRRRRRLKAQADIYKAR | 15 | 4181.65 | 4181.60 +/− 0.69 | B-CRRRRRRRLKAQADIYKARFQAERQAR | 16 |
| | FQAERHAREK | | | | EK | |
| A-Cterm-LZ | RQIKIWFQNRRMKWKKLVEKK | 20 | | | RQIKIWFQNRRMKWKKLAEKKELLQEQLE | 21 |
| | EYLQEQLEQLQREFNKL | | | | QLQREYSKL | |
| spCC2-LZ | LEDLRQQLQQAEEALVAKQEL | 17 | | | LEDLKQQLQQAEEALVAKQEVIDKLKEEA | 19 |
| | IDKLKEEAEQHKIVMETVPVL | | | | EQHKIVMETVPVLKAQADIYKADFQAERQ | |
| | KAQADIYKADFQAERHAREKL | | | | AREKLAEKKELLQEQLEQLQREYSKL | |
| | VEKKEYLQEQLEQLQREFNKL | | | | | |
| BA-LZ | B-CRQIKIWFQNRRMKWKKLK | 22 | | | | |
| L322P/L329P | AQADIYKADFQAERHAREKLV | | | | | |
| | EKKEYPQEQLEQPQREFNKL | | | | | |
| FLAG-NEMO | MDYKDDDDKDPPISRGSEFLD | 25 | | | | |
| | NKHPWKNQLSEMVQPSGGPAE | | | | | |
| | DQDMLGEESSLGKPAMLHLPS | | | | | |
| | EQGTPETLQRCLEENQELRDA | | | | | |
| | IRQSNQMLRERCEELLHFQVS | | | | | |
| | QREEKEFLMCKFQEARKLVER | | | | | |
| | LSLEKLDLRSQREQALKELEQ | | | | | |
| | LKKCQQQMAEDKASVKVQVTS | | | | | |
| | LLGELQESQSRLEAATKDRQA | | | | | |
| | LEGRIRAVSEQVRQLESEREV | | | | | |
| | LQQQHSVQVDQLRMQNQSVEA | | | | | |
| | ALRMGRQAASEEKRKLAQLQA | | | | | |
| | AYHQLFQDYDSHIKSNKGMQL | | | | | |
| | EDLRQQLQQAEEALVAKQELI | | | | | |
| | DKLKEEAEQHKIVMETVPVLK | | | | | |
| | AQADIYKADFQAERHAREKLV | | | | | |
| | EKKEYLQEQLEQLQREFNKLK | | | | | |
| | VGCHESARIEDMRKRHVETPQ | | | | | |
| | PTLLPAPAHHSFHLALSNQRR | | | | | |
| | SPPEEPPDFCCPKCQYQAPDM | | | | | |
| | DTLQIHVMECIE | | | | | |
| GFP-NEMO | MVSKGEELFTGVVPILVELDG | 27 | | | | |
| | DVNGHKFSVSGEGEGDATYGK | | | | | |
| | LTLKFICTTGKLPVPWPTLVT | | | | | |
| | TLTYGVQCFSRYPDHMKQHDF | | | | | |
| | FKSAMPEGYVQERTIFFKDDG | | | | | |
| | NYKTRAEVKFEGDTLVNRIEL | | | | | |
| | KGIDFKEDGNILGHKLEYNYN | | | | | |
| | SHNVYIMADKQKNGIKVNFKI | | | | | |
| | RHNIEDGSVQLADHYQQNTPI | | | | | |
| | GDGPVLLPDNHYLSTQSALSK | | | | | |
| | DPNEKRDHMVLLEFVTAAGIT | | | | | |
| | LGMDELYKEFLDNKHPWKNQL | | | | | |
| | SEMVQPSGGPAEDQDMLGEES | | | | | |
| | SLGKPAMLHLPSEQGTPETLQ | | | | | |
| | RCLEENQELRDAIRQSNQMLR | | | | | |
| | ERCEELLHFQVSQREEKEFLM | | | | | |
| | CKFQEARKLVERLSLEKLDLR | | | | | |
| | SQREQALKELEQLKKCQQQMA | | | | | |
| | EDKASVKVQVTSLLGELQESQ | | | | | |
| | SRLEAATKDRQALEGRIRAVS | | | | | |
| | EQVRQLESEREVLQQQHSVQV | | | | | |
| | DQLRMQNQSVEAALRMGRQAA | | | | | |
| | SEEKRKLAQLQAAYHQLFQDY | | | | | |
| | DSHIKSNKGMQLEDLRQQLQQ | | | | | |
| | AEEALVAKQELIDKLKEEAEQ | | | | | |
| | HKIVMETVPVLKAQADIYKAD | | | | | |
| | FQAERHAREKLVEKKEYLQEQ | | | | | |
| | LEQLQREFNKLKVGCHESARI | | | | | |
| | EDMRKRHVETPQPTLLPAPAH | | | | | |
| | HSFHLALSNQRRSPPEEPPDF | | | | | |
| | CCPKCQYQAPDMDTLQIHVME | | | | | |
| | CIE | | | | | |
| Biot-CC2-LZ | Biotin⁽²⁾- | 28 | 10503,123 | 503.27 +/− 0.79 | Biotin⁽²⁾- | 29 |
| | LEDLRQQLQQAEEALVAKQEL | | | | LEDLRQQLQQAEEALVAKQELIDKLKEEA | |
| | IDKLKEEAEQHKIVMETVPVL | | | | EQHKIVMETVPVLKAQADIYKADFQAERQ | |
| | KAQADIYKADFQAERHAREKL | | | | AREKLAEKKELLQEQLEQLEREYSKL⁽³⁾ | |
| | VEKKEYLQEQLEQLQREFNKL | | | | | |
| His-CC2-LZ | MGSHHHHHHSSGLVPRGSHMA | 30 | 10503,123 | 503.27 +/− 0.79 | MGSHHHHHHGLVPRGSHMASLEDLRQQLQ | 31 |
| | SLEDLRQQLQQAEEALVAKQE | | | | QAEEALVAKQELIDKLKEEAEQHKIVMET | |
| | LIDKLKEEAEQHKIVMETVPV | | | | VPVLKAQADIYKADFQAERQAREKLAEKK | |
| | LKAQADIYKADFQAERHAREK | | | | ELLQEQLEQLQREYSKL⁽³⁾ | |
| | LVEKKEYLQEQLEQLQREFNKL | | | | | |

⁽¹⁾In peptides coupled to Bodipy, the N-terminus contains a cysteine residue for convenience of specific peptide coupling with the maleimide group as described in the Patent Application WO2005/027959. The sequences of antennapedia and poly-arginine (R₇) fused to the NEMO sequence (plain text) arehighlighted in bold characters. Residues that may be involved in coiled-coil sequence are underlined and those that were replaced in the CC2 and LZ mutants and in NLM-DR are underlined in bold characters. B = Bodipy (The C-terminal Bodipy modification has been omittedfrom the sequences appearing in the Sequence Listing). Within the scope of the present invention it is contemplated that Bodipy and/or the N-terminal cysteine may be removed and used as described herein.
⁽²⁾Biotin, also known as vitamin H or B₇, has the chemical formula C₁₀H₁₆N₂O₃S and is a water-soluble B-complex vitamin which is composed of an ureido (tetrahydroimidizalone) ring fused with a tetrahydrothiophene ring. A valeric acid substituent is attached to one of the carbon atoms of the tetrahydrothiophene ring.
⁽³⁾Residues in bold are residues which are present in the human CC2-LZ.

### C.—Conditions for FACS Analysis

The internalization of the peptides is analyzed by FACS as described in the International Application WO 2005/027959.

### D.—NF-κB Inhibition Assays

4.5×10⁵ 70Z/3-C3 cells in 450 μl of 10% FCS-RPMI (Invitrogen) were incubated with 0-20 μM peptide BA-DR NLM or BA-NLM. After 2 h at 37° C., 200 μl of cell samples were transferred in duplicate to a 96-well micro titer plate. One well was treated for 4 h with either 15 μg/ml LPS, 20 ng/ml IL1-β or 100 ng/ml PMA. A control well was left untreated. After 4 h at 37° C., cells were centrifuged at 400×g for 5 min at room temperature, the pellets were washed three times with cold PBS (200 μl), and the cells were lysed in 100 μl of lysis buffer (25 mM tris-phosphate buffer at pH7.8 containing 8 mM MgCl₂, 1 mM dithioerythreitol, 1% Triton X-100, 15% glycerol and a protease inhibitor mixture (Roche Applied Science, ref. 1836145)). The lysate was centrifuged at 1000×g for 20 min at 4° C., and supernatant was kept on ice before performing the β-galactosidase assay. β-galactosidase assays were performed on 50 μl of the supernatant and in an assay mix containing 4 μl of Galacton-star chemiluminescent substrate and 196 μl of reaction buffer (Clonetech). The activity was measured with a plate luminometer (Berthold).

### E.—Inhibition Assays of Other Signalisation Pathways

Jurkat T cells (clone 20) were transiently transfected by a DEAE-dextran method with a reporter plasmid containing the luciferase under the control of a promoter inducible by the ERK and p38 pathway (SRE-luc; Courtois et al., 1997, Mol. Cell. Biol, 17:1441-1449), the NF-AT pathway or the API pathway (respectively NF-AT-luc and AP1-luc; Northrop et al., 1993, J. Biol. Cell., 268:2917-2923). Briefly, cells were washed and resuspended at 10⁷ cells/ml in TBS containing 0.5 mg/ml of DEAE-dextran (Pharmacia). 1 μg per 2·10⁶ cells of reporter plasmid was added. After 45 min at room temperature, the cells were diluted with 10 volumes of TBS, centrifuged, and resuspended at 10⁶ cells/ml in 10% FCS-RPMI. After 24 h, cells were incubated with or without 5 μM NEMO-derived peptides (BA-NLM or BA-DR NLM) for 2 h. They were stimulated or mock-stimulated with 100 ng/ml PMA and 1 μg/ml ionomycin for 5 h, and finally lysed in luciferase buffer (25 mM Tris-phosphate pH 7.8 containing 8 mM MgCl₂, 1 mM DTE, 1% Triton X100, 15% glycerol). Luciferase measurement was carried out in a Berthold Luminometer.

### F.—Fluorescence Anisotropy

Anisotropy measurements were performed with a QuantaMaster® fluorometer (PTI), equipped with polarizing filters for excitation and emission, and using a photomultiplier tube in the L-configuration. All experiments were carried out in a microcuvette (60 μl) at 22° C., with excitation and emission wavelengths at 495 and 513 nm. The band pass of the excitation and emission wavelengths was 2 and 4 nm, respectively. Steady-state fluorescence anisotropy, expressed as millianisotropy units was measured as described Agou et al., 2004a. All measurements were carried out in a 20 mM isoionic buffer pH 8 (10 mM acetic acide, 10 mM MES, 20 mM Tris), containing 20 mM KCl. Each data point is the result of 20 recording taken over a 2-min period.

### G.—Circular Dichroism Spectroscopy

Far-UV circular dichroism (CD) measurements were performed with an Aviv 215 spectropolarimeter with samples diluted in 10 mM NaPO₄ at pH 7.

### H.—Identification of Proteins Bound to Bodipy-Conjugated Peptides

5·10⁶ JM4.5.2/Flag-NEMO cells were seeded in 6-well plates (3 ml/well) and incubated with 20 μM of Bodipy-conjugated peptides (BA-NLM or BA-DR NLM) for 4 h at 37° C. Cells were centrifuged at 400×g for 5 min, resuspended in 200 μl of an hypotonic buffer (10 mM TrisHCl pH 7.9, 1.5 mM MgCl₂, 10 mM KCl), left on ice for 20 min and lysed by several passage through a 26 G needle. Lysates were clarified at 15 000×g for 20 min and 80 μg of total proteins were incubated with 40 μl of agarose anti-Flag M2 beads for 1 h at 4° C. The Flag-NEMO protein were pulled down with the agarose anti-Flag M2 beads by centrifugation for 1 min. Beads were resuspended in 100 μl of hypotonic buffer and the fluorescence of NEMO linked peptide was read on microplate reader from SAFAS. NEMO coated beads were analyzed by Western blotting using anti-Flag M2 monoclonal antibodies to normalize the sample using the UN-SCAN-IT software.

### I.—Inhibition of NEMO Oligomerization in Cells

293T cells were transiently co-transfected by calcium-phosphate-DNA precipitation method with 2 μg of a plasmid pcDNA3 (Invitrogen) containing the sequence of FLAG-NEMO (pcDNA3-Flag-NEMO of sequence SEQ ID NO: 24) and 0.7 μg of a plasmid pcDNA3 containing the sequence of GFP-NEMO (pcDNA3-GFP-NEMO of sequence SEQ ID NO: 26). Empty vector is added up to a total amount of DNA of 4 μg per 6 cm dish. 24 h after transfection, cells were resuspended and incubated with or without 20 μM of peptides (A-NLM or A-DR NLM) for 7 h at 37° C. Cells were resuspended and washed twice with PBS to remove excess peptides and lysed in 150 μl of lysis buffer containing a protease inhibitor mixture as indicated above (Example 1.D). Lysates were clarified at 15 000×g at 4° C. for 20 min. The amount of proteins in the clarified lysate is determined by the Bradford method with the kit DC Biorad. 100 μg of total proteins were diluted in 200 μl of interaction buffer (50 mM TrisHCl pH 7.5, 1% triton X100, 1 mM DTE, 300 mM NaCl) and incubated 1 h at 4° C. with 40 μl of agarose anti-Flag M2 beads (Sigma). The oligomers Flag-NEMO/GFP-NEMO were pulled down with the anti-Flag M2 beads and washed twice with the interaction buffer. The fluorescence of GFP-NEMO were directly read on the beads with a fluorescence microplate reader SAFAS and the clarified lysates were analysed by Western blotting using anti-Flag, anti-GFP and anti-NDPK-B antibodies (Kraeft et al., 1996; Exp Cell Res; 227:63-69) to normalize the results.

J.—Identification of Polyubiquitinylated Proteins Bound to the Peptide spCC2-LZ Comprising the CC2 and LZ Domains of NEMO.

24·10⁷ JM4.5.2 cells were stimulated or not with 10 ng/ml of TNFα for 10 minutes at 37° C., washed twice with cold PBS and lysed in 900 μl of lysis buffer containing a protease inhibitor mixture as indicated in Example 1.D. Lysates were clarified at 15 000×g at 4° C. for 30 min. 150 μl of proteins were incubated 2 h at 4° C. with 150 μl of streptavidin magnetic beads (MagPrep®, Novagen) pre-incubated 1 h at 4° C. with 10 μg of biotynilated-CC2-LZ and with or without 20 μM of peptides (BA-NLM or BA-DR NLM). Beads were washed 4 times in buffer A (10 mM Hepes pH 7.5, 150 mM NaCl, 8 mM MgCl₂, 10% glycerol, 0.1 mM DDM and 1 mM DTE) and resuspended in 30 μl of laemmli buffer containing 6 M Urea. Samples were analysed by Western blotting using anti-ubiquitin antibodies (Sigma) as well as anti-NDPK-B antibodies for loading controls.

K.—Search for Compounds that Compete for CC2-LZ Dimerization Induced by the Interaction with Polyubiquitin Chains

The Homogeneous Time Resolved Fluorescence (HTRF®) assay reagents anti-(His)₆ monoclonal antibody labeled with XL665 (MABHis₆-XL) and streptavidin-Europium cryptate (streptavidin-EuK) were purchased from CIS-Bio. The His-CC2-LZ peptide (SEQ ID NO: 30 and 31) was purified as described in Agou et al., 2004a and the biotin tagged CC2-LZ peptide (SEQ ID NO: 28 and 29) was chemically synthezised as described in Wyler et al., 2007.

Monoubiquitin and K63-linked polyubiquitin chains were purchased from Boston Biochem. The cDNA encoding the linear tetraubiquitin fused to its terminus with the GST protein is a gift of Y. Dikic. The GST-Ub₄ was purified using Glutathione Sepharose 4 Fast Flow following manufacter's instructions (GE Healthcare). All measurements of fluorescence resonance energy transfer (FRET) were performed using the microplate reader Mithras LB 940 (Berthold Technologies). HTRF® principle

In HTRF experiments, the pure His-CC2-LZ protein was labeled by anti-His-XL665 mAb and biot-CC2-LZ was labeled by streptavidin-EuK. Dimerization of His-CC2-LZ and Biot-CC2-LZ leads the donor (Steptavidin-EuK) and the acceptor (anti-His-XL665) into close proximity. Upon excitation of Europium cryptate at 320 nm, FRET occurs between these two fluorophores, and XL665 re-emits a specific long-lived fluorescence at 665 nm.

All experiments were carried out in a black microplate Greiner (200 μl) at 25° C. The excitation wavelength was 320 nm and the fluorescence emissions were measured at 620 nm and at 665 nm to normalize the specific signal. Fluorescence emission was recorded at both wavelengths after an 50 μs delay, which reduces background noise introduced by the media and the free acceptor molecules. The FRET signal is calculated using the following equation:

ΔF=[(R−R_{neg})/R_{neg}]*100 (1)

where R is the ratio ([emission at 665 nm/emission at 620 nm]*10 000) calculated for each assay and R_{neg} is the same ratio for the negative control (i.e. in the absence of His-CC2-LZ).

Briefly, the His-CC2-LZ and biot-CC2-LZ (50 nM each) proteins were mixed with each other in the binding buffer 20 mM potassium phosphate pH 7.0 containing 100 mM potassium fluoride and 0.1% BSA. Following incubation, the Streptavidin-EuK (donor) and the antibody Anti-His-XL665 (acceptor) were added in the mixture at concentrations of 2 nM and 7 nM respectively. For the negative control, all components described above were present in the mixture except the His-CC2-LZ which was replaced by the binding buffer. Under these conditions, no FRET signal above background could be detected.

### Example 2

### DR NLM Peptide Binds In Vitro to the Oligomerization Domain of NEMO

Previous studies of circular dichroism and gel filtration show that the NLM and DR NLM peptides do not form oligomeric coiled-coil structures and behave as monomer at a peptide concentration up to 100 μM (data not shown). Then, unlike the LZ peptide, DR NLM peptide as well as NLM peptide are unable to self-associate. Self association of LZ peptide is described by Agou et al., 2004b.

Here the inventors confirm that DR NLM inhibits the NF-kB pathway after activation of the pre-B 70Z3 lymphocytes with a phorbol ester (PMA), a pro-inflammatory cytokine (IL-1) and an endotoxin (LPS) (FIGS. 1A and 1B). The IC₅₀ measured for these various stimuli are similar (0.9 μM+/−0.1). Moreover, the use of a variety of internalization peptides (Tat, Antennapedia, R7, R9) yields similar IC₅₀ values, suggesting that internalization is not the limiting step.

To analyse whether DR NLM binds the MOD of NEMO, fluorescent labelled BA-DR NLM (10 μM) was incubated with an increasing concentrations of spCC2-LZ and the complex formation was monitored by fluorescence polarization (P) and results are expressed as anisotropy (A) according to the formula A=2P/(3-P). The concentration of spCC2-LZ was determining by amino acid analysis (Vinolo et al; 2006). Data points were fitted to the binding isotherm equation which gives a dissociation constant of 17 μM. Inset, the same experiment was done with BR₇-DR NLM (10 μM) which gives a similar dissociation constant (K_{D}=21 μM).

DR NLM form a stable MOD/peptide complex with an affinity of 17 μM and a stoichiometry of 3:1 (FIG. 2A). This suggests that DR NLM peptide interacts preferentially with the trimer of MOD. This interaction does not depend on the cell permeable sequence because DR NLM fused to its N-terminus with the antennapedia peptide (BA-DR NLM) or the poly R₇ peptide (BR₇-DR NLM) displays similar dissociation constants (Inset, FIG. 2A). The binding site is located by fluorescence polarization using various peptides that mimic several regions of MOD (FIG. 2B). This site corresponds to the C-terminal part of NEMO's LZ subdomain (residues 315-336). This site was previously determined to be essential for NEMO function through point mutation analysis (Agou et al., 2004a).

These studies probing the selectivity of DR NLM-induced NF-κB inhibition in various cell types capture the most attractive properties of the peptide.

Results of the inhibition assays described in Example 1.E are depicted in FIG. 3. They demonstrate that NLM-DR does not abrogate at least four other pathways including AP1, ERK, p38 and NF-AT in T-lymphocytes in responses to PMA and ionomycin, indicating that the DR NLM peptide, but not the NLM peptide, inhibits specifically the NF-κB pathway (FIG. 3).

### Example 3

### DR NLM Directly Interacts with NEMO in Cells

To address the question whether the inhibition observed by the DR NLM peptide was due to a direct interaction with NEMO, we used NEMO-deficient T lymphocytes (JM4.5.2 cell lines) stably reconstituted with the gene of Flag-NEMO protein (JM4.5.2/Flag-NEMO) as described in Example 1.A. After incubation of the cells with BA-NLM-DR fluorescent labelled peptide (20 μM) or the fluorescent labelled NLM control (BA-LZ L322P/L329P) for 4 h, the cells were lysed and the crude extracts were incubated with anti-Flag antibodies covalently linked to agarose beads as described in Example 1.H (see FIG. 4A for an overview of the method). Following the pull-down of the beads, the fluorescence attached to the beads was measured, (Example 1.H), providing an assay of the amount of peptide bound to NEMO (FIGS. 4A and 4B). This assay was normalized relative to the expression level of Flag-NEMO protein in each sample by Western blot using anti-Flag M2 mono-clonal antibody. FIG. 4B shows that DR NLM binds specifically to NEMO, showing a perfect correlation between the effect of a given peptide in inhibiting the activation of the pathway and its ability to interact with NEMO.

### Example 4

### DR NLM does not Interfere with Nemo Oligomerization

In order to better analyze the molecular mechanism of the inhibition of the NF-κB pathway by DR NLM peptide, the Inventors have developed an assay to probe NEMO oligomerization in cells as described in Example 1.I (see FIG. 5 for an overview of the cell-based assay). For this, two plasmids were used, expressing respectively GFP-NEMO and Flag-NEMO fusion proteins. The plasmids were cotransfected in human 293T cells to overexpress exogenous GFP-NEMO and Flag-NEMO fusion proteins and enforce NEMO oligomerization. The cells were then submitted to detergent lysis and Flag-NEMO proteins were immunopurified using anti-Flag agarose beads and Flag peptide as described in Example 1.I. The fluorescence associated with beads that results from an association of Flag-NEMO with GFP-NEMO subunits within hetero-oligomers allow an estimate of the amount of oligomers present in the cellular extracts. Controls including the expression of GFP-NEMO alone or the co-expression of Flag-NEMO and GFP proteins do not show any fluorescence associated with the anti-Flag beads (FIG. 6A).

The BA-LZ peptide inhibits in vitro NEMO oligomerization (Agou et al., 2004b). Then, BA-LZ was used as positive control to validate the above fluorescence cell-based assay. Under the conditions of this assay, the fluorescence emission of Bodipy does not interfere with the measure of the fluorescence emission of GFP. As shown in FIG. 6B, the incubation with BA-LZ peptide (20 μM) results in a strong decrease in the amount of fluorescent NEMO subunit, indicating that the BA-LZ peptide inhibits NEMO oligomerization in cells. In contrast, when a homologous peptide (BA-LZ L322P/L329P) bearing a double mutation within the coiled-coil interface is used, or when the cells are treated without peptide, a higher amount of the fluorescent NEMO subunit was recovered. Interestingly the formation of NEMO oligomers was equivalent in the absence or in the presence of the mutant peptide (BA-LZ L322P/L329P) indicating that no dissociation of NEMO oligomers was observed with this mutant peptide.

Using this cell based assay, the inventors have examined the capacity of the peptide BA-DR NLM to alter NEMO oligomerization. FIG. 6C shows the same amount of NEMO oligomers in the absence or in the presence of BA-DR NLM or BA-NLM peptides (20 μM), despite the ability of BA-DR NLM to bind to the NEMO protein. In conclusion, in cells experiments point to a new mechanism of NEMO inhibition independent of its state of oligomerization.

### Example 5

### DR NLM Inhibits in Cells the Interaction Between CC2-LZ and Polyubiquitinylated proteins

Recent reports showed that NEMO also acts as a sensor of Lys 63-linked polyubiquitination (Ea et al., 2006; Wu et al., 2006). Thus, the Inventors examined whether DR NLM impedes the interaction between NEMO and the K63 polyubiquitin chains. To this end, they developed a pull down assay which uses the a peptide comprising the CC2 and the LZ domains of NEMO as a bait to pull down the polyubiquitinylated proteins as described in Example 1.J and depicted in FIG. 7A. The Biotinylated-CC2-LZ (Bio-CC2-LZ) coated on magnetic beads was co-incubated in the absence or in the presence of A-NLM or A-DR NLM peptides with crude extracts from NEMO deficient T lymphocytes (JM4.5.2 cell lines) stimulated or not with TNFα. After extensive washing of beads, all mono and polyubiquitinylated proteins bound to CC2-LZ were analysed by Western blot using anti-ubiquitin antibody (Sigma). As shown in FIG. 7B, DR NLM peptide abolishes the CC2-LZ interaction with the cellular polyubiquitinylated proteins in a TNF-α dependent manner while no inhibition was observed in the absence or in the presence of the NLM peptide. These results show a significant loss of interaction between polyubiquitinylated proteins and CC2-LZ, indicating that DR NLM peptide inhibits the NF-κB pathway by blocking the interaction between NEMO and the polyubiquitinylated proteins. Correlatively, in vitro binding assays using fluorescence polarization show that the dissociation constant and the cooperativity index (Hill coefficient) of tag-free DR NLM for the Lys63-linked polyubiquitin chains (K_{D}=250 μM, n=1) are lower than those of tag-free NLM (K_{D}=180 μM, n=8). This indicates that the NLM peptide of NEMO represents a new ubiquitin binding motif and that the simple mutation D→R in the motif leads to a complete loss of interaction of NEMO with polyubiquitin chains.

The results outlined above and combined with those previously reported in the international Patent Application W02005027959 show that the region of NEMO comprising the CC2 and LZ domains acts as oligomerization domain as well as polyubiquitin chain binding domain. These dual properties of NEMO domain may allow to search for new compounds inhibiting the NF-κB signaling pathway by disrupting NEMO oligomerization, using a screening assay as described in Examples 1.11a), b) and c), or by disrupting the interaction between NEMO and the polyubiquitin chains, using a screening assay as described in Example 1.12.

### Example 6

### Screening Assays Based on CC2-LZ Oligomerization

A.—Search for Compounds that Compete for LZ Peptide Binding to the Peptide CC2-LZ Comprising the CC2 and LZ Domains of NEMO

Anisotropy measurements are performed with a SAFAS Xenius fluorometer. All experiments are carried out in a black microplate Greiner (100 μl) at 20° C. with excitation and emission wavelengths at 495 and 515 nm. The band pass of the excitation and emission wavelengths is 8 nm. R₇-LZ Bodipy labeled (BR₇-LZ) and R₇-LZ peptides are indicated in Table I and the recombinant His-CC2-LZ protein is purified as described by Agou et al., 2004b.

BR₇-LZ (5 μM) and R₇-LZ (20 μM) are incubated at 25° C. with the target His-CC2-LZ (10 μM) to induce the formation of a stable complex in 100 μl of Tris/MES buffer (10 mM acide acétique, 10 mM MES) at pH 8 containing 50 mM KCl. The non-fluorescent peptide which shows the same affinity as the fluorescent peptide allows to increase the amount of peptides linked to the target. Under these experimental conditions, the addition of His-CC2-LZ leads to an anisotropy increase of 20% relative to the anisotropy signal of free R₇-NLM-LZ*BR₇-LZ peptide (77 mA) indicating the formation of a complex His-CC2-LZ:BR₇-LZ. The His-CC2-LZ/R₇-LZ/BR₇-LZ mixture is then seeded on 96-well plastic plates containing drug compound candidates. Hits are selected for their capacity to decrease fluorescence anisotropy, reflecting the peptide dissociation from His-tag CC2-LZ target.

B.—Search for Compounds that Induce a Conformational Switch Toward an Inactive Conformation of the Peptide CC2-LZ Comprising the CC2 and LZ Domains of NEMO

To develop this screening assay, Fluorescence Resonance Energy Transfer method (FRET) is used. The CC2-LZ peptide is conjugated at its N-terminus with a fluorophore donor such as Cy5/5 (GE Healthcare) and at its C-terminus with a fluorophore acceptor like BHQ3 (Biosearch Technologies). The doubly conjugated CC2-LZ is brought into contact with a molecule to be tested and the fluorescence is measured. Hits that induce an increase of fluorescence emission will be selected.

C.—Search for Compounds that Inhibits CC2-LZ Oligomerization

A screening assay which relies on Homogeneous time resolved fluorescence (HTRF) will also be set up for a higher throughput. Europium-Cryptate (fluorophore donor) and XL665 (fluorophore acceptor) conjugated antibodies that respectively recognize the biotin and the (His)₆ tag are commercially available (Cis-Bio inc.) The purification of His-tag CC2-LZ peptide is described in Agou et al., 2004b. The biotin tagged CC2-LZ peptide is chemically synthezised or is purified from a recombinant E. coli strain such as AviTag (Avidity, inc). The main benefit of this method is that it is affortable and it diminishes the risk of false positive

### Example 7

### Screening Assays Based on Polyubiquitin Binding Properties of the CC2 and LZ Domains

A.—Primary Screening Assay

In a first step K63 linked polyubiquitin chains (K63Ub₂₋₇, Boston Biochem, Inc.) is immobilized on Ni-NTA HisSord plates (96 well plates, Qiagen, Inc). The fluorescent labelled CC2-LZ (e.g. B-CC2-LZ) or the fluorescent NLM peptide (e.g. B-DR NLM) is added to form fluorescent protein complexes with the K63 polyubiquitin chains. Hits that compete for the CC2-LZ or the NLM binding to K63 polyubiquitin chains is considered as positive and is selected.

B.—Secondary Screening Assay

In a second step, all positive hits that derived from primary screenings described above are submitted to a secondary screening to examine their effects on NF-κB inhibition using the cell-based assay which was described in the W02005027959 patent.

Abbreviations used: BA-peptide, peptide fused to its N-terminus with the cell permeable sequence antennapedia and conjugated with the bodipy fluorophor; A-peptide, Antennapedia tagged peptide; R7-peptide, peptide fused to its terminus with the cell permeable polycationic peptide RRRRRRR; Tat, cell permeable peptide containing the sequence YGRKKRRQRRR; R9, cell permeable cationic peptide containing nine arginine residues; spCC2-LZ, synthetic polypeptide subdomain CC2-LZ; DDM, dodecyl maltoside; MES, morpholinoethane sulfonic acid; DTE, dithio-erythrol.

### Example 8

### Search for Compounds that Compete for CC2-LZ Dimerization Induced by the Interaction with Polyubiquitin Chains

The Inventors also used the well-characterized complexes of the CC2-LZ dimer bound to Designed Ankyrin Repeat Proteins (DARPins) (Wyler et al., 2007). These complexes display nM affinities for the CC2-LZ dimer and were used as positive controls. In this assay, the His-tagged protein CC2-LZ was replaced either by the His-tagged DARPin 1D5 (K_{D}=22 nM) or by the His-tagged DARPin 2F6 (K_{D}=8 nM). As shown in the Table II, a FRET signal was observed with 1D5 (290%) and 2F6 (2749%) DARPins, demonstrating the efficacy of the FRET assay for identifying nM protein complexes.

TABLE II
Negative and positive controls used in the HTRF assay

| Strept- | AntiHis- | Biot- | His-CC2- | | | |
|---|---|---|---|---|---|---|
| Eu | XL | CC2-LZ | LZ | His-1DS | His-2F6 | FRET |
| (2 nM) | (7 nM) | (50 nM) | (50 nM) | (50 nM) | (50 nM) | (ΔF %) |
| + | + | − | − | − | − | ≦0 |
| + | + | + | − | − | − | ≦0 |
| + | + | − | + | − | − | ≦0 |
| + | + | + | + | − | − | ≦0 |
| + | + | + | − | + | − | 290% |
| + | + | + | − | − | + | 2749% |

Different combinations of protein samples as indicated by (+) and (−), were used to measure the fluorescence resonance energy transfer (FRET) in the assay. The percentage of FRET was calculated as described in Example 1 above.

Since the affinity of CC2-LZ dimerization is low (K_{D}=22 μM) as compared to those of DARPin:CC2-LZ complexes, no FRET signal was observed when each subunit of CC2-LZ was incubated at nM concentration (see Table II). However, when the same mixture was incubated in the presence of GST-tetraubiquitine, a specific signal of FRET was monitored, suggesting that the tetraubiquitine binding induces tha formation of CC2-LZ dimer. Similar experiments were performed with monoubiquitin or with GST alone. However, no FRET signal was detected either with the GST alone or with the monoubiquitin (FIG. 8 and Table III), indicating that the FRET signal results from the specific interaction of CC2-LZ dimer with GST-tetraubiquitin chains.

TABLE III
Effects of GST-Ub₄ binding on the formation of CC2-LZ dimmers.

| Strept- | AntiHis- | Biot-CC2- | His-CC2- | | | | |
|---|---|---|---|---|---|---|---|
| Eu | XL | LZ | LZ | Mono-Ub | Poly-Ub | GST-Ub₄ | FRET |
| (2 nM) | (7 nM) | (50 nM) | (50 nM) | (1 μM) | (12.5 μM) | (12.5 μM) | (ΔF %) |
| + | + | + | + | + | − | − | ≦0 |
| + | + | + | + | − | + | − | ≦0 |
| + | + | + | + | − | − | + | 373% |

In order to optimize the HTRF assay, the inventors also used variable concentrations of His-CC2-LZ, Biot-CC2-LZ and of GST-Ub₄. Results shown in FIGS. 8A and 8B indicate that the optimal conditions for a robust and significant FRET signal in HTRF experiments correspond to 40 nM Biot-CC2-LZ, 40 nM His-CC2-LZ and 250 nM GST-Ub₄. Moreover, the inventors have shown that the presence of DMSO up to 5% does not disturb the fluorescent signal (FIG. 8C).

In conclusion, the inventors have developed a powerful and robust FRET assay that will allow the searching for small compounds that compete for the CC2-LZ dimerisation and/or for the specific binding of CC2-LZ dimer to polyubiquitin chains.

### REFERENCES

Agou, F., Ye, F., Goffinont, S., Courtois, G., Yamaoka, S., Israel, A. and Véron, M. (2002) NEMO trimerizes through its coiled-coil C-terminal domain. J. Biol. Chem., 20, 17464-17465.
Agou, F., Traincard, F., Vinolo, E., Courtois, G., Yamaoka, S., Israel, A. and Veron, M. (2004a) The trimerization domain of NEMO is composed of the interacting C-terminal CC2 and LZ coiled-coil subdomains. J Biol Chem, 279, 27861-27869.
Agou, F., Courtois, G., Chiaravalli, J., Baleux, F., Coic, Y. M., Traincard, F., Israel, A. and Veron, M. (2004b) Inhibition of NF-kappa B activation by peptides targeting NF-kappa B essential modulator (nemo) oligomerization. J Biol Chem, 279, 54248-54257.
Carter R S, Geyer B C, Xie M, Acevedo-Suarez C A, Ballard D W. (2001) Persistent activation of NF-kappa B by the tax transforming protein involves chronic phosphorylation of IkappaB kinase subunits IKKbeta and IKKgamma. J Biol. Chem. July 6; 276(27):24445-8. Epub 2001 Apr. 26.
Chan D C and Kim P S. (1998) HIV entry and its inhibition. Cell. 1998 May 29; 93(5):681-4. Review. No abstract available.
Courtois G, Whiteside S T, Sibley C H, Israel A. (1996) J Biol Cell 268: 2917-2923.
Mike Chang, Lianshan Zhang, James P. Tam, and Elaine Sanders-Bush (2000) Dissecting G Protein-coupled Receptor Signaling Pathways with Membrane-permeable Blocking Peptides. J. Biol. Chem., March 2000; 275: 7021-7029.
Döffinger, R., Smahi, A., Bessia, C., Geissman, F., Feinberg, J., Durandy, A., Bodemer, C., Kenwrick, S., Dupuis-Girod, S., Blanche, S., Wood, P., Rabia, S. H., Headon, D. J., Overbeek, P. A., Le Deist, F., Holland, S., Belani, K., Kumararatne, D. S., Fisher, A., Shapiro, R., Conley, M. E., Reimund, E., Kalhoff, H., Abinun, M., Munnich, A., Israel, A., Courtois, G. and Casanova, J.-L. (2001) X-linked anhidrotic ectodermal dysplasia with immunodeficiency is caused by impaired NF-κB signaling. Nature Genet., 27, 277-285.
Ea, C. K., Deng, L., Xia, Z. P., Pineda, G. and Chen, Z. J. (2006) Activation of IKK by TNFalpha requires site-specific ubiquitination of RIP1 and polyubiquitin binding by NEMO. Mol Cell, 22, 245-257.
Eckert, D. M., and Kim, P. S. (2001) Mechanisms of viral membrane fusion and its inhibition. Ann. Rev. Biochem., 70, 777-810
Erickson et al., Biophys J., 2003, 85, 599-611
Ghosh, S., May, M. J. and Kopp, E. B. (1998) NF-κB and Rel proteins: evolutionarily conserved mediators of immune responses. Annu. Rev. Immunol., 16, 225-260.
Jin Gohda, Masato Irisawa, Yuetsu Tanaka, Shintaro Sato, Kiyoshi Ohtani, Jun-ichi Fujisawa and Jun-ichiro Inoue (2007) HTLV-1 Tax-induced NFκB activation is independent of Lys-63-linked-type polyubiquitination. Biochemical and Biophysical Research Communications, 357: 1, 225-230.
Harhaj, E. W., Good, L., Xiao, G., Uhlik, M., Cvijic, M. E., Rivera-Walsh, I. and Sun, S. C. (2000) Somatic mutagenesis studies of NF-kappa B signaling in human T cells: evidence for an essential role of IKK gamma in NF-kappa B activation by T-cell costimulatory signals and HTLV-I Tax protein. Oncogene, 19, 1448-1456.
Yinling H U, Veronique BAUD, Takefumi O G A, Keun I L KIM, Kazuhiko YOSHIDA and Michael KARIN (2001) IKKγ controls formation of the epidermis independently of NF-κB Nature 410, 710-714
Inohara, N., Koseki, T., Lin, J., del Peso, L., Lucas, P. C., Chen, F. F., Ogura, Y. and Nunez, G. (2000) An induced proximity model for NF-κB activation in the Nod1/RICK and RIP signaling pathways. J. Biol. Chem., 275, 27823-27831.
Israel, A. (2000) The IKK complex: an integrator of all signals that activate NF-κB? Trends Cell. Biol., 10, 129-133.
J. Kevin Judice, Jeffrey Y. K. Tom, Wei Huang, Terri Wrin, Joann Vennari, Christos J. Petropoulos, and Robert S. McDowell (1997) Inhibition of HIV type 1 infectivity by constrained α-helical peptides: Implications for the viral fusion mechanism PNAS 94: 13426-13430.
Karawawa et al., Biochem J., 2004, 381(Pt1), 307-12
Karin, M. (1999) The beginning of the end: IκB Kinase (IKK) and NF-κB activation. J. Biol. Chem., 274, 27339-27342.
Kovalenko, A., Chable-Bessia, C., Cantarella, G., Israël, A., Wallach, D. and Courtois, G. (2003) The tumor suppressor CYLD negatively regulates NF-κB signalling by deubiquitination. Nature, 424, 801-805.
Kraeft S K, Traincart F, Mesnildrey S, Bourdais J, Veron M, Chen L B (1996) Exp Cell Res, 227: 63-69.
Yao-Zhong Lin, SongYi Yao, Ruth Ann Veach, Troy R. Torgerson, and Jacek Hawiger (1995) Inhibition of Nuclear Translocation of Transcription Factor NF-κB by a Synthetic Peptide Containing a Cell Membrane-permeable Motif and Nuclear Localization Sequence. J. Biol. Chem., June 1995; 270: 14255-14258.
May, M. J. et al. (2000) Selective inhibition of NF-κB activation by a peptide that blocks the interaction of NEMO with the IκB kinase complex. Science, 289, 1550-1554
May, M. J., Marienfield, R. B. and Ghosh, S. (2002) Characterization of the IκB-kinase NEMO binding domain. J. Biol. Chem., 277, 45992-46000.
Northrop J P, Ullman K S, Crabtree G R (1993) J Biol Chem 268: 2917-2923.
Pawson, T. and Nash, P. (2003) Assembly of cell regulatory systems through protein interaction domains. Science, 300, 445-452.
Joel L. Pomerantz and David Baltimore. (2002) Two Pathways to NF-κB Molecular Cell 2002 10: 693-695
Poulaki V., Mitsiades C. S., Joussen A. M., Lappas A., Kirchhof B., Mitsiades N. (2002) Constitutive nuclear factor-kappaB activity is crucial for human retinoblastoma cell viability. Am J Pathol., 161, 2229-2240.
Poyet, J.-L., Srinivasula, S. M., Lin, J-H, Fernandes-Alnmeri, T., Yamaoka, S., Tsichlis, P. N. and Alnemri, E. S. (2000) Activation of yhe IκB kinases by RIP via IKKγ/NEMO-mediated oligomerization. J. Biol. Chem., 275, 37966-37977.
Poyet, J.-L., Srinivasula, S. M. and Alnemri, E. S. (2001) vClap, a caspase-recruitment domain-containing protein of equine Herpesvirus-2, persistently activates the IκB kinases through oligomerization of IKKγ. J. Biol. Chem., 276, 3183-3187
Santoro, G., Rossi, A. and Amici, C. (2003) NF-κB and virus infection: who controls whom. EMBO J., 22, 2552-2560.
Uwe Senftleben, Yixue Cao, Gutian Xiao, Florian R. Greten, Gertraud Krähn, Giuseppina Bonizzi, Yi Chen, Yinling Hu, Abraham Fong, Shao-Cong Sun, and Michael Karin. (2001) Activation by IKK of a Second, Evolutionary Conserved, NF-κB Signaling Pathway. Science August 24; 293: 1495-1499.
Shaner et al., Nat. Biotech., 2004, 22, 1567.
Souroujon M. C. and Mochly-Rosen D. (1998) Peptide modulators of protein-protein interactions in intracellular signaling. Nat. Biotechnol., 16, 919-924.
Eric D. Tang, Naohiro Inohara, Cun-Yu Wang, Gabriel Nuñez, and Kun-Liang Guan. (2003) Roles for homotypic interactions and transautophosphorylation in B kinase (IKK) activation. J Biol. Chem. Vol. 278, 38566-38570
Tegethoff, S. et al. (2003) Tetrameric oligomerization of IκB kinase γ is obligatory for IKK complex activity and NF-κB activation. Mol. Cell. Biol., 23, 2029-2041
Trompouki, E., Hatzivassilou, E., Tsichritzis, T., Farmer, H., Ashworth, A. and Mosialos, G. (2003) CYLD is a deubiquitinating enzyme that negatively regulates NF-κB activation by TNFR family members. Nature, 424, 793-796
Vinolo E, Sebban H, Chaffotte A, Israel A, Courtois G, Veron M, Agou F (2006) J Biol Chem 281:6334-48.
Wyler et al., 2007 Inhibition of NF-kB activation with designed ankyrin-repeat proteins targeting the ubiquitin-binding/oligomerization domain of NEMO. Protein Sci 16(9), p 2013-2022.
Wu, C. J., Conze, D. B., Li, T., Srinivasula, S. M. and Ashwell, J. D. (2006) Sensing of Lys 63-linked polyubiquitination by NEMO is a key event in NF-kappaB activation. Nat Cell Biol, 8, 398-406.
Ebrahim Zandi, David M. Rothwarf, Mireille Delhase, Makio Hayakawa, and Michael Karin. (1997) The IκB Kinase Complex (IKK) Contains Two Kinase Subunits, IKKα and IKKβ, Necessary for IκB Phosphorylation and NF-κB Activation. Cell 91: 243-252.
Honglin Zhou, Ingrid Wertz, Karen O'Rourke, Mark Ultsch, Somasekar Seshagiri, Michael Eby, Wei Xiao, Vishva M. Dixit (2004) Bcl10 activates the NF-κB pathway through ubiquitination of NEMO. Nature 427, 167-171.
Zonana, J., Elder, M. E., Schneider, L. C., Orlow, S. J., Moss, C., Golabi, M., Shapira, S. K., Farndon, P. A., Wara, D. W., Emmal, S. A. and Ferguson, B. M. (2000) A novel X-linked disorder of immune deficiency and hypohydrotic ectodermal dysplasia is allelic to incontinentia pigmenti and due to mutations in IKK-γ (NEMO). Am. J. Hum. Genet. 67, 1555-1562.

1. An in vitro method for primary screening for a potential modulator of the NF-κB pathway activation, steps said method comprising:
(a) bringing into contact (i) a peptide P1 consisting of the CC2 and LZ regions of NEMO, said peptide P1 being labelled or not, and (ii) a peptide P2, labelled or not, selected from the group consisting of a peptide comprising at least 10 amino acid residues of the LZ region of NEMO, a peptide comprising at least 10 amino acid residues of the NLM region of NEMO, a peptide comprising DR-NLM, a peptide comprising the CC2 and LZ regions of NEMO, a peptide comprising at least 10 amino acid residues of the CC2 region of NEMO, and a peptide comprising a K63-linked polyubiquitinylated chain; in the absence of the substance S to be tested;
(b) bringing into contact said peptide P1, labelled or not, and said peptide P2, labelled or not, in the presence of the substance S to be tested;
(c) detecting the complexes between P1 and P2 obtained in (a) by measuring an appropriate signal;
(d) detecting the complexes between P1 and P2 obtained in (b) by measuring an appropriate signal; and
(e) comparing the signal measured in (c) and the signal measured in (d) and selecting the substance S for which the ratio of the signal measured in (c)/signal measured in (d) is different from 1.

2. The method according to claim 1, wherein P1 is selected from the group consisting of the peptides of sequences SEQ ID NO: 1, 2, 17, 19, 28, 29, 30, 31.

3. The method according to claim 1, wherein P2 is selected from the group consisting of the peptides of sequences SEQ ID NO: 1, SEQ ID NO: 2, SEQ ID NO: 6 to 12, SEQ ID NO: 17 to 19, SEQ ID NO: 28 to 31.

4. The method according to claim 1, wherein said peptide P1 is immobilized on a solid support.

5. The method according to claim 4, wherein said peptide P1 is coupled to a biotin group and said solid support consists of streptavidin beads.

6. The method according to claim 1, wherein the signal in (c) and (d) is measured by Western blot, by fluorescent anisotropy, by Fluorescent Resonance Energy Transfer (FRET) or by Homogeneous Time Resolved Fluorescence (HTRF).

7. The method according to claim 1, wherein said peptide P1 is unlabelled and said peptide P2 is labelled with a fluorescent marker M1.

8. The method according to claim 7, wherein said marker M1 is selected from the group consisting of: green fluorescent protein (GFP), cyan fluorescent protein, yellow fluorescent protein and a fluorophore comprising a maleimide group.

9. The method according to claim 7, wherein said peptide P2 labelled with said marker M1 is selected from the group consisting of the peptides of sequences SEQ ID NO: 4, 5 and 13 to 16.

10. The method according to claim 1, wherein said peptide P1 is labelled with a marker M2 and said peptide P2 is labelled with a marker M3, M2 and M3 being a couple of a fluorophore donor and a fluorophore acceptor.

11. The method according to claim 10, wherein the marker M2 consists of a first tag and an antibody directed against said first tag and labelled with a fluorophore donor, and the marker M3 consists of a second tag and an antibody directed against said second tag and labelled with a fluorophore acceptor, said two tags being different.

12. The method according to claim 11, wherein said tag of the marker M2 is a poly His sequence, and said tag of the marker M3 is biotin.

13. The method according to claim 1, wherein said potential modulator is a potential activator and in that (e) comprises comparing the signal measured in (c) and the signal measured in (d) and selecting the substance S for which the ratio of the signal measured in (c)/signal measured in (d) is less than 1.

14. The method according to claim 1, wherein said potential modulator is a potential inhibitor and in that (e) comprises comparing the signal measured in (c) and the signal measured in (d) and selecting the substance S for which the ratio of the signal measured in (c)/signal measured in (d) is greater than 1.

15. A method for primary screening for a potential modulator of the NF-κB pathway activation, said method comprising:
(a) bringing into contact (i) a peptide P1 selected from the group consisting of: a peptide comprising at least 10 amino acid residues of the NLM region of NEMO, and a peptide comprising DR NLM, said peptide P1 being labelled or not, and (ii) a peptide P2 comprising a K63-linked polyubiquitine chain, said peptide P2 being labelled or not, in the absence of the substance S to be tested;
(b) bringing into contact said peptide P1, labelled or not, and said peptide P2, labelled or not, in the presence of the substance S to be tested;
(c) detecting the complexes between P1 and P2 obtained in (a) by measuring an appropriate signal;
(d) detecting the complexes between P1 and P2 obtained in (b) by measuring an appropriate signal; and
(e) comparing the signal measured in (c) and the signal measured in (d) and selecting the substance for which the ratio of the signal measured in (c)/signal measured in (d) is different from 1.

16. The method according to claim 15, wherein said peptide P1 is selected from the group consisting of the peptides of sequences SEQ ID NO: 1, 2, 10 and 17 to 19.

17. The method according to claim 15, wherein said peptide P2, comprising a K63-linked polyubiquitinylated chain, is immobilised on a solid support.

18. The method according to claim 15, wherein said potential modulator is a potential activator and in that (e) comprises comparing the signal measured in (c) and the signal measured in (d) and selecting the substance S for which the ratio of the signal measured in (c)/signal measured in (d) is less than 1.

19. The method according to claim 15, wherein said potential modulator is a potential inhibitor and in that (e) comprises comparing the signal measured in (c) and the signal measured in (d) and selecting the substance S for which the ratio of the signal measured in (c)/signal measured in (d) is greater than 1.

20. A method for primary screening for a potential modulator of the NF-κB pathway activation, said method comprising:
(a) bringing into contact a peptide P1 comprising the CC2 and LZ regions of NEMO with the substance S to be tested, said peptide P1 being labelled with the markers M2 a fluorophore donor and M3 a fluoropohore acceptor;
(b) measuring the fluorescence emission in the absence of the substance S to be tested;
(c) measuring the fluorescence emission in the presence of the substance S to be tested; and
(d) comparing the signal measured in (b) and the signal measured in (c) and selecting the substance S for which the ratio signal measured in (b)/signal measured in (c) is different from 1.

21. The method according to claim 20, wherein said peptide P1 is selected from the peptides of sequences SEQ ID NO: 1, 2, 17, 19, 28, 29, 30, 31.

22. The method according to claim 20, wherein said fluorophore donor M2 is coupled to the N-terminus of the CC2 domain, and said fluorophore acceptor M3 is coupled to the C-terminus of the LZ domain.

23. The method according to claim 20, wherein said potential modulator is a potential activator and in that (d) comprises comparing the signal measured in (b) and the signal measured in (c) and selecting the substance S for which the ratio of the signal measured in (b)/signal measured in (c) is less than 1.

24. The method according to claim 20, wherein said potential modulator is a potential inhibitor and in that (d) comprises comparing the signal measured in (b) and the signal measured in (c) and selecting the substance S for which the ratio of the signal measured in (b)/signal measured in (c) is greater than 1.

25. A method for primary screening for a potential modulator of NF-κB pathway activation, said method comprising:
a) providing at least one cell deficient in endogenous NEMO, which is transformed with one or more polynucleotides that express at least a peptide P1 and a peptide P2 as defined in claim 1;
b) applying the substance S to said at least one cell;
c) measuring the formation of complexes between P1 and P2 in the absence of the substance S;
d) measuring the formation of complexes between P1 and P2 in the presence of the substance S; and
e) comparing the signal measured in (c) and the signal measured in (d) and by selecting the substance S for which the ratio of the signal measured in (c)/signal measured in (d) is different from 1.

26. The method according to claim 25, wherein the peptide P1 is a fusion protein GFP-NEMO and the peptide P2 is a fusion protein Flag-NEMO.

27. The method according to claim 26, wherein (c) and (d) comprise:
c1) (or d1)) the cell lysis,
c2) (or d2)) the immunoprecipitation of Flag-NEMO, bound or not to GFP-NEMO, using antibody directed against the Flag moiety of Flag-NEMO, and
c3) (or d3)) the detection of the fluorescence emission of the GFP moiety of GFP-NEMO bound to the immunoprecipitated Flag-NEMO.

28. The method according to claim 25, wherein said potential modulator is a potential activator and in that (e) comprises comparing the signal measured in (c) and the signal measured in (d) and selecting the substance S for which the ratio of the signal measured in (c)/signal measured in (d) is less than 1.

29. The method according to claim 25, wherein said potential modulator is a potential inhibitor and in that (e) comprises comparing the signal measured in (c) and the signal measured in (d) and selecting the substance S for which the ratio of the signal measured in (c)/signal measured in (d) is greater than 1.

30. A method for secondary screening for an inhibitor of NF-κB pathway activation, comprising:
a) selecting a substance S potentially able to inhibit NF-κB pathway activation by a method according to claim 14,
b) applying an activator of NF-κB pathway in the absence or in the presence of the substance S selected in a), to a cell comprising a reporter gene under the control of a promoter inducible by NF-κB;
c) detecting the expression of the reporter gene by measuring an appropriate signal in the absence of the substance S;
d) detecting the expression of the reporter gene by measuring an appropriate signal in the presence of the substance S; and
e) comparing the signal measured in (c) and the signal measured in (d) and selecting the substance for which the ratio of the signal measured in (e)/signal measured in (d) is greater than 1.

31. The method for secondary screening for an activator of NF-κB pathway activation, comprising:
a) selecting a substance S potentially able to activate NF-κB pathway activation by a method according to claim 13,
b) providing a cell comprising a reporter gene under the control of a promoter inducible by NF-κB;
c) detecting the expression of the reporter gene by measuring an appropriate signal in the absence of the substance S;
d) detecting the expression of the reporter gene by measuring an appropriate signal in the presence of the substance S; and
e) comparing the signal measured in (c) and the signal measured in (d) and selecting the substance for which the ratio of the signal measured in (c)/signal measured in (d) is less than 1.

32. The method according to claim 30, wherein said activator of NF-κB is selected from the group consisting of: TNF-α, IL-1β, PMA, ionomycin, and LPS from Salmonella abortus.

33. The method according to claim 30, wherein said reporter gene is the gene lacZ encoding the β galactosidase or the gene luc encoding the luciferase.

34. The method according to claim 30, wherein said cell is a 70Z/3-C3 cell, deposited in the Collection Nationale de Cultures de Microorganismes (C.N.C.M.) on Apr. 1, 2003, under the number I-3004.


SEQUENCE LISTING (35 sequences)

SEQ ID NO: 1 - protein, 412 aa, Mus musculus
MNKHPWKNQLSETVQPSGGPAEDQDMLGEESSLGKPAMLHLPSEQGTPETLQRCLEENQELRDAIRQSNQMLRERCEELLHFQVSQREEKEFLMCKFQEARKLVERLSLEKLDLRSQREQALKELEQLKKCQQQMAEDKASVKAQVTSLLGELQESQSRLEAATKDRQALEGRIRAVSEQVRQLESEREVLQQQHSVQVDQLRMQNQSVEAALRMERQAASEEKRKLAQLQAAYHQLFQDYDSHIKSSKGMQLEDLRQQLQQAEEALVAKQELIDKLKEEAEQHKIVMETVPVLKAQADIYKADFQAERHAREKLVEKKEYLQEQLEQLQREFNKLKVGCHESARIEDMRKRHVETPQPPLLPAPAHHSFHLALSNQRRSPPEEPPDFCCPKCQYQAPDMDTLQIHVMECIE

SEQ ID NO: 2 - protein, 419 aa, Homo sapiens
MNRHLWKSQLCEMVQPSGGPAADQDVLGEESPLGKPAMLHLPSEQGAPETLQRCLEENQELRDAIRQSNQILRERCEELLHFQASQREEKEFLMCKFQEARKLVERLGLEKLDLKRQKEQALREVEHLKRCQQQMAEDKASVKAQVTSLLGELQESQSRLEAATKECQALEGRARAASEQARQLESEREALQQQHSVQVDQLRMQGQSVEAALRMERQAASEEKRKLAQLQVAYHQLFQEYDNHIKSSVVGSERKRGMQLEDLKQQLQQAEEALVAKQEVIDKLKEEAEQHKIVMETVPVLKAQADIYKADFQAERQAREKLAEKKELLQEQLEQLQREYSKLKASCQESARIEDMRKRHVEVSQAPLPPAPAYLSSPLALPSQRRSPPEEPPDFCCPKCQYQAPDMDTLQIHVMECIE

SEQ ID NO: 3 - protein, 17 aa, Artificial Sequence
  Description of Artificial Sequence Synthetic BA peptide
CRQIKIWFQNRRMKWKK

SEQ ID NO: 4 - protein, 60 aa, Artificial Sequence
  Description of Artificial Sequence Synthetic m BA-LZ polypeptide
CRQIKIWFQNRRMKWKKLKAQADIYKADFQAERHAREKLVEKKEYLQEQLEQLQREFNKL

SEQ ID NO: 5 - protein, 60 aa, Artificial Sequence
  Description of Artificial Sequence Synthetic h BA-LZ polypeptide
CRQIKIWFQNRRMKWKKLKAQADIYKADFQAERQAREKLAEKKELLQEQLEQLQREYSKL

SEQ ID NO: 6 - protein, 43 aa, Artificial Sequence
  Description of Artificial Sequence Synthetic m LZ polypeptide
LKAQADIYKADFQAERHAREKLVEKKEYLQEQLEQLQREFNKL

SEQ ID NO: 7 - protein, 43 aa, Artificial Sequence
  Description of Artificial Sequence Synthetic h LZ polypeptide
LKAQADIYKADFQAERQAREKLAEKKELLQEQLEQLQREYSKL

SEQ ID NO: 8 - protein, 40 aa, Artificial Sequence
  Description of Artificial Sequence Synthetic m CC2 polypeptide
SKGMQLEDLRQQLQQAEEALVAKQELIDKLKEEAEQHKIV

SEQ ID NO: 9 - protein, 40 aa, Artificial Sequence
  Description of Artificial Sequence Synthetic h CC2 polypeptide
SKGMQLEDLKQQLQQAEEALVAKQEVIDKLKEEAEQHKIV

SEQ ID NO: 10 - protein, 21 aa, Artificial Sequence
  Description of Artificial Sequence Synthetic m NLM peptide
LKAQADIYKADFQAERHAREK

SEQ ID NO: 11 - protein, 21 aa, Artificial Sequence
  Description of Artificial Sequence Synthetic m DR NLM peptide
LKAQADIYKARFQAERHAREK

SEQ ID NO: 12 - protein, 21 aa, Artificial Sequence
  Description of Artificial Sequence Synthetic h DR NLM peptide
LKAQADIYKARFQAERQAREK

SEQ ID NO: 13 - protein, 38 aa, Artificial Sequence
  Description of Artificial Sequence Synthetic m BA-DR NLM polypeptide
CRQIKIWFQNRRMKWKKLKAQADIYKARFQAERHAREK

SEQ ID NO: 14 - protein, 38 aa, Artificial Sequence
  Description of Artificial Sequence Synthetic h BA-DR NLM polypeptide
CRQIKIWFQNRRMKWKKLKAQADIYKARFQAERQAREK

SEQ ID NO: 15 - protein, 29 aa, Artificial Sequence
  Description of Artificial Sequence Synthetic m BR7-DR NLM peptide
CRRRRRRRLKAQADIYKARFQAERHAREK

SEQ ID NO: 16 - protein, 29 aa, Artificial Sequence
  Description of Artificial Sequence Synthetic h BR7-DR NLM peptide
CRRRRRRRLKAQADIYKARFQAERQAREK

SEQ ID NO: 17 - protein, 84 aa, Artificial Sequence
  Description of Artificial Sequence Synthetic CC2-LZ polypeptide
LEDLRQQLQQAEEALVAKQELIDKLKEEAEQHKIVMETVPVLKAQADIYKADFQAERHAREKLVEKKEYLQEQLEQLQREFNKL

SEQ ID NO: 18 - protein, 21 aa, Artificial Sequence
  Description of Artificial Sequence Synthetic h NLM peptide
LKAQADIYKADFQAERQAREK

SEQ ID NO: 19 - protein, 84 aa, Artificial Sequence
  Description of Artificial Sequence Synthetic h CC2-LZ polypeptide
LEDLKQQLQQAEEALVAKQEVIDKLKEEAEQHKIVMETVPVLKAQADIYKADFQAERQAREKLAEKKELLQEQLEQLQREYSKL

SEQ ID NO: 20 - protein, 38 aa, Artificial Sequence
  Description of Artificial Sequence Synthetic murine A-Cterm LZ polypeptide
RQIKIWFQNRRMKWKKLVEKKEYLQEQLEQLQREFNKL

SEQ ID NO: 21 - protein, 38 aa, Artificial Sequence
  Description of Artificial Sequence Synthetic h A-Cterm-LZ polypeptide
RQIKIWFQNRRMKWKKLAEKKELLQEQLEQLQREYSKL

SEQ ID NO: 22 - protein, 60 aa, Artificial Sequence
  Description of Artificial Sequence Synthetic BA-LZ L322P/L329P polypeptide
CRQIKIWFQNRRMKWKKLKAQADIYKADFQAERHAREKLVEKKEYPQEQLEQPQREFNKL

SEQ ID NO: 23 - protein, 8 aa, Artificial Sequence
  Description of Artificial Sequence Synthetic flag peptide
DYKDDDDK

SEQ ID NO: 24 - DNA, 2787 bp, Artificial Sequence
  Description of Artificial Sequence Synthetic pcDNA3-Flag-NEMO polynucleotide
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagaccctagaactagtggatccgccaccatggactacaaggacgacgatgacaaagatcccccgatctctcgaggatccgaattcctcgacaacaagcacccctggaagaaccagctgagtgagatggtgcagcccagtggtggcccagcagaggaccaggacatgctgggtgaagaatcttctctggggaagcctgcaatgctacatctgccttcagagcagggtactcctgagaccctccagcgctgcctggaagagaatcaagagctccgagacgctatccggcagagcaatcagatgctgagggaacgctgtgaggagctgctgcatttccaggtcagccagcgggaggagaaggagttccttatgtgcaaattccaggaagcccggaagctggtggagagactgagcttggagaagcttgatcttcggagtcagagggaacaggccttaaaggagttggagcaactgaagaaatgccaacagcagatggctgaggacaaggcctctgtgaaagttcaggtgacatcattgctcggagaactccaggagagccagagccgtttggaggctgccaccaaggatcggcaagctttagagggaaggattcgagcagttagtgagcaggtcagacagctggagagtgagcgggaggtgctacagcagcagcacagcgtccaggtggaccagctgcgtatgcagaaccagagcgtggaggctgccttgcgaatggggcggcaggctgcttcagaggagaagcggaagctggctcagttgcaggcagcctatcaccagctcttccaagactacgacagccacattaagagcaacaagggcatgcagctggaagatctgaggcaacagctccagcaagctgaggaggccctggtagccaaacaggaattgattgataagctgaaagaggaggctgagcagcacaagattgtgatggagactgtgccagtcttgaaggcccaggcggatatctacaaggctgacttccaagctgagaggcatgcccgggagaagctggtggagaagaaggagtatttgcaggagcagctggagcagctgcagcgcgagttcaacaagctgaaagttggctgccatgagtcagccaggattgaggatatgaggaagcggcatgtagagactccccagcctactttactccctgctccagctcaccactcctttcatttggccttgtccaaccagcggaggagccctcctgaagaacctcctgacttctgttgtccgaagtgccagtatcaggctcctgatatggacactctacagatacatgtcatggagtgcatagagtaggggcagcagatgcaaggccacttgcagtactatgtcctgatctgtgtgacttgtgctttcctgttttacctgcatagtccacacttaagggcttgctttagccctttggtcccccatttagggtagacagccccattcagggcttttttttttttctgtgtgcctgatccagtttgcctctggtggcttcttccctcttctcccatagtcctagggagtctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggc

SEQ ID NO: 25 - protein, 432 aa, Artificial Sequence
  Description of Artificial Sequence Synthetic pcDNA3-Flag-NEMO polypeptide
MDYKDDDDKDPPISRGSEFLDNKHPWKNQLSEMVQPSGGPAEDQDMLGEESSLGKPAMLHLPSEQGTPETLQRCLEENQELRDAIRQSNQMLRERCEELLHFQVSQREEKEFLMCKFQEARKLVERLSLEKLDLRSQREQALKELEQLKKCQQQMAEDKASVKVQVTSLLGELQESQSRLEAATKDRQALEGRIRAVSEQVRQLESEREVLQQQHSVQVDQLRMQNQSVEAALRMGRQAASEEKRKLAQLQAAYHQLFQDYDSHIKSNKGMQLEDLRQQLQQAEEALVAKQELIDKLKEEAEQHKIVMETVPVLKAQADIYKADFQAERHAREKLVEKKEYLQEQLEQLQREFNKLKVGCHESARIEDMRKRHVETPQPTLLPAPAHHSFHLALSNQRRSPPEEPPDFCCPKCQYQAPDMDTLQIHVMECIE

SEQ ID NO: 26 - DNA, 3541 bp, Artificial Sequence
  Description of Artificial Sequence Synthetic pcDNA3-GFP-NEMO polynucleotide
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttgcttacatttgcttctgacacaactgtgttcactagcaacctcaaacagacaccatgagatcttatccatatgatgtgccagattatgcagccatggccggatccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggaattcctcgacaacaagcacccctggaagaaccagctgagtgagatggtgcagcccagtggtggcccagcagaggaccaggacatgctgggtgaagaatcttctctggggaagcctgcaatgctacatctgccttcagagcagggtactcctgagaccctccagcgctgcctggaagagaatcaagagctccgagacgctatccggcagagcaatcagatgctgagggaacgctgtgaggagctgctgcatttccaggtcagccagcgggaggagaaggagttccttatgtgcaaattccaggaagcccggaagctggtggagagactgagcttggagaagcttgatcttcggagtcagagggaacaggccttaaaggagttggagcaactgaagaaatgccaacagcagatggctgaggacaaggcctctgtgaaagttcaggtgacatcattgctcggagaactccaggagagccagagccgtttggaggctgccaccaaggatcggcaagctttagagggaaggattcgagcagttagtgagcaggtcagacagctggagagtgagcgggaggtgctacagcagcagcacagcgtccaggtggaccagctgcgtatgcagaaccagagcgtggaggctgccttgcgaatggggcggcaggctgcttcagaggagaagcggaagctggctcagttgcaggcagcctatcaccagctcttccaagactacgacagccacattaagagcaacaagggcatgcagctggaagatctgaggcaacagctccagcaagctgaggaggccctggtagccaaacaggaattgattgataagctgaaagaggaggctgagcagcacaagattgtgatggagactgtgccagtcttgaaggcccaggcggatatctacaaggctgacttccaagctgagaggcatgcccgggagaagctggtggagaagaaggagtatttgcaggagcagctggagcagctgcagcgcgagttcaacaagctgaaagttggctgccatgagtcagccaggattgaggatatgaggaagcggcatgtagagactccccagcctactttactccctgctccagctcaccactcctttcatttggccttgtccaaccagcggaggagccctcctgaagaacctcctgacttctgttgtccgaagtgccagtatcaggctcctgatatggacactctacagatacatgtcatggagtgcatagagtaggggcagcagatgcaaggccacttgcagtactatgtcctgatctgtgtgacttgtgctttcctgttttacctgcatagtccacacttaagggcttgctttagccctttggtcccccatttagggtagacagccccattcagggcttttttttttttctgtgtgcctgatccagtttgcctctggtggcttcttccctctctcccatagtcctagggagtctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggc

SEQ ID NO: 27 - protein, 654 aa, Artificial Sequence
  Description of Artificial Sequence Synthetic pcDNA3-GFP-NEMO polypeptide
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKEFLDNKHPWKNQLSEMVQPSGGPAEDQDMLGEESSLGKPAMLHLPSEQGTPETLQRCLEENQELRDAIRQSNQMLRERCEELLHFQVSQREEKEFLMCKFQEARKLVERLSLEKLDLRSQREQALKELEQLKKCQQQMAEDKASVKVQVTSLLGELQESQSRLEAATKDRQALEGRIRAVSEQVRQLESEREVLQQQHSVQVDQLRMQNQSVEAALRMGRQAASEEKRKLAQLQAAYHQLFQDYDSHIKSNKGMQLEDLRQQLQQAEEALVAKQELIDKLKEEAEQHKIVMETVPVLKAQADIYKADFQAERHAREKLVEKKEYLQEQLEQLQREFNKLKVGCHESARIEDMRKRHVETPQPTLLPAPAHHSFHLALSNQRRSPPEEPPDFCCPKCQYQAPDMDTLQIHVMECIE

SEQ ID NO: 28 - protein, 84 aa, Artificial Sequence; identical to SEQ ID NO: 17
  Description of Artificial Sequence Synthetic Biot-CC2-LZ polypeptide

SEQ ID NO: 29 - protein, 84 aa, Artificial Sequence
  Description of Artificial Sequence Synthetic Biot-CC2-LZ humanised polypeptide
LEDLRQQLQQAEEALVAKQELIDKLKEEAEQHKIVMETVPVLKAQADIYKADFQAERQAREKLAEKKELLQEQLEQLQREYSKL

SEQ ID NO: 30 - protein, 106 aa, Artificial Sequence
  Description of Artificial Sequence Synthetic His-CC2-LZ polypeptide
MGSHHHHHHSSGLVPRGSHMASLEDLRQQLQQAEEALVAKQELIDKLKEEAEQHKIVMETVPVLKAQADIYKADFQAERHAREKLVEKKEYLQEQLEQLQREFNKL

SEQ ID NO: 31 - protein, 106 aa, Artificial Sequence
  Description of Artificial Sequence Synthetic His-CC2-LZ humanised polypeptide
MGSHHHHHHSSGLVPRGSHMASLEDLRQQLQQAEEALVAKQELIDKLKEEAEQHKIVMETVPVLKAQADIYKADFQAERQAREKLAEKKELLQEQLEQLQREYSKL

SEQ ID NO: 32 - protein, 6 aa, Artificial Sequence
  Description of Artificial Sequence Synthetic 6xHis tag
HHHHHH

SEQ ID NO: 33 - protein, 7 aa, Artificial Sequence
  Description of Artificial Sequence Synthetic peptide
RRRRRRR

SEQ ID NO: 34 - protein, 9 aa, Artificial Sequence
  Description of Artificial Sequence Synthetic peptide
RRRRRRRRR

SEQ ID NO: 35 - protein, 11 aa, Artificial Sequence
  Description of Artificial Sequence Synthetic peptide
YGRKKRRQRRR